(19) United States
(12) Patent Application Publication
(10) Pub. No.: US 2022/0180971 A1
(43) Pub. Date: Jun. 9, 2022

(54) COMPUTER-IMPLEMENTED METHOD FOR COLLABORATIVE DEVELOPMENT OF REAGENTS FOR DETECTION OF TARGET NUCLEIC ACIDS

(71) Applicant: SEEGENE, INC., Seoul (KR)

(72) Inventors: Jong Yoon CHUN, Yeoju-si (KR); Dae Hoon LEE, Seoul (KR); Yun Jee KIM, Seoul (KR); Youn Chul CHOI, Hanam-si (KR); Young Wook KIM, Seoul (KR)

(21) Appl. No.: 17/439,900

(22) PCT Filed: Jun. 12, 2020
(86) PCT No.: PCT/KR2020/007664

§ 371 Date: Sep. 16, 2021

(30) Foreign Application Priority Data
Jun. 14, 2019 (KR) 10-2019-0070900
Dec. 27, 2019 (KR) 10-2019-0177063

(51) Int. Cl. G16B 35/00; G16B 40/20; G06Q 10/10

(52) U.S. Cl. CPC G16B 35/00; G06Q 10/101; G16B 40/20

(57) ABSTRACT

The disclosure relates to a computer-implemented method, system, and storage medium for collaborative development of a reagent for detection of the target nucleic acid. According to the disclosure, the method, system, and storage medium automatically match the developer and technology provider suitable for the characteristics of collaborative development in response to a request for collaborative development, thereby increasing the efficiency of collaborative development.

### CROSS-REFERENCE TO RELATED APPLICATIONS

This application claims priority from Korean Patent Application No. 10-2019-0070900, filed on Jun. 14, 2019, Korean Patent Application No. 10-2019-0177063, filed on Dec. 27, 2019, in the Korean Intellectual Property Office, the disclosure of which is incorporated herein by reference in its entirety.

### TECHNICAL FIELD

The disclosure relates to computer-implemented methods, systems, and storage media for collaborative development of reagents for detection of target nucleic acids.

### BACKGROUND ART

Nucleic acid amplification is a pivotal process for a wide variety of methods in molecular biology, such that various amplification methods have been proposed. For example, Miller, H. I. et al. (WO 89/06700) discloses a method for amplifying nucleic acids, comprising the hybridization of a promoter/primer sequence to a target single-stranded DNA (“ssDNA”) followed by transcription of many RNA copies of the sequence. Other known nucleic acid amplification procedures include transcription-based amplification systems (Kwoh, D. et al., Proc. Natl. Acad. Sci. U.S.A., 86:1173(1989); and Gingeras T. R. et al., WO 88/10315).

The most predominant process for a nucleic acid amplification known as polymerase chain reaction (hereinafter referred to as “PCR”) is based on repeated cycles of denaturation of double-stranded DNA, followed by oligonucleotide primer annealing to the DNA template, and primer extension by a DNA polymerase (Mullis et al. U.S. Pat. Nos. 4,683,195, 4,683,202, and 4,800,159; Saiki et al., (1985) Science 230, 1350-1354).

PCR-based techniques have been widely used not only for amplification of a target DNA sequence, but also for scientific applications or methods in the fields of biological and medical research, such as reverse transcriptase PCR (RT-PCR), dif-ferential display PCR (DD-PCR), cloning of known or unknown genes by PCR, rapid amplification of cDNA ends (RACE), arbitrary priming PCR (AP-PCR), multiplex PCR, SNP genome typing, and PCR-based genomic analysis (McPherson and Moller, (2000) PCR. BIOS Scientific Publishers, Springer-Verlag New York Berlin Heidelberg, NY).

The above-listed PCR-based techniques amplify and detect a target nucleic acid to determine the presence or absence of the target nucleic acid. In particular, if the target nucleic acid is associated with a specific disease (e.g., when the target nucleic acid originates from a particular pathogen), the presence of the target nucleic acid can help diagnose the disease in a subject.

Such PCR-based techniques need use of a reagent for detection of a target nucleic acid, including an oligonucleotide (e.g., a primer and/or probe) which is hybridized specifically to the target nucleic acid, a label, DNA polymerase, dNTPs, Mg ions, and buffer so as to amplify and detect the target nucleic acid.

However, the development and commercialization of such reagent essentially requires sophisticated design of oligonucleotides and complex performance tests for the designed oligonucleotide, thus calling for significant time and efforts, know-how, and costs. It is not easy to acquire clinical samples for the performance tests, causing it more difficult to develop a reagent.

Furthermore, the development of reagents for simultaneously detecting multiple target nucleic acids in one reaction requires much more labor and costs due to an increased number of oligonucleotides, as compared with reagents for detection of a single target nucleic acid. For such reasons, reagents for simultaneous detection of multiple target nucleic acids have been produced by only a few manufacturers.

It is common that reagent developers own a development technology limited to a specific field (e.g., such as for detection of a target nucleic acid in a human sample) and that reagents are difficult to develop for use in other fields, e.g., for detection of a target nucleic acid in an animal, plant, or food sample.

To overcome the hurdles in developing a reagent, there have been attempts for collaborative developments among some groups, but it has been unsuccessful due to difficulty in finding/matching proper collaborative partner and lack of systematic management or low efficiency of collaborative development.

The tropics or jungles where the annual mean temperature is high suffer from the outbreaks of various hereditary or infectious diseases or region-specific epidemics but these regions do not see any advance in development of a reagent for molecular diagnosis due to poor technology. Huge multinational molecular diagnostic reagent manufacturers mostly located in developed countries are unwilling to develop products for diagnosing region-specific epidemics due to low profits.

A need exists for a new method and system for addressing such issues.

Throughout this application, various patents and publications are referenced and citations are provided in parentheses. The disclosure of these patents and publications in their entirety are hereby incorporated by references into this application in order to more fully describe this invention and the state of the art to which this invention pertains.

### DISCLOSURE OF INVENTION

### Technical Problem

The inventors have endeavored to come up with a method or system for developing various reagent for detection of the target nucleic acids in a more efficient and simultaneous manner. As a result, the inventors have found that various reagents for detection of the target nucleic acids may be developed simulataneously and without regional limitations via collaboration between a technology provider and a developer.

According to the present disclosure, the developers located in the disease outbreak region may develop a reagent for detection of a target nucleic acid associated with the disease by receiving technical know-how from technology provider via collaborative development, and the reagent thus developed may be used in the disease outbreak region.

After developing in one region, an infectious disease may spread to other regions where the target nucleic acid associated with the disease may then be mutated and, for this reason, development of a reagent for detecting the target nucleic acid needs to be hastened. The developers in the disease outbreak region may quickly develop and use a reagent for detection of the target nucleic acid via collaborative development by which they may receive technology providers' know-how.

It is an object of the present invention to provide a computer-implemented method for collaborative development of a reagent for detection of the target nucleic acid.

It is another object of the present invention to provide a system for collaborative development of a reagent for detection of the target nucleic acid.

It is still another object of the present invention to provide a computer-readable storage medium including instructions for collaborative development of a reagent for detection of the target nucleic acid.

Other objects and advantages of the present invention will become apparent from the detailed description to follow taken in conjugation with the appended claims and drawings.

### Solution to Problem

In an aspect of the disclosure, there is provided computer-implemented method for collaborative development of a reagent for detection of a target nucleic acid by a collaborative development system, comprising: receiving from a requester terminal a request for collaborative development of a reagent containing an oligonucleotide for detection of a target nucleic acid, wherein the request for collaborative development includes feature information about the target nucleic acid to be detected using the reagent; allowing a developer terminal to access a technology provider's development tool kit, wherein the development tool kit includes a performance test tool for the oligonucleotide to be included in the reagent, the performance test tool including one or more of software, a device, and instructional information; receiving from the developer terminal a result obtained from using the development tool kit, wherein the result includes performance data for the oligonucleotide; allowing the received result to be reviewed by the technology provider terminal or by a monitoring module of a collaborative development system; and allowing the developer terminal to access the review product, such that an oligonucleotide to be included in the reagent is selected.

In other aspect of the disclosure, there is provided a system for collaborative development of a reagent for detection of the target nucleic acid using one or more programs stored in a memory and configured to be executed by a processor, wherein the one or more programs include instructions executed by the processor to perform: receiving from a requester terminal a request for collaborative development of a reagent containing an oligonucleotide for detection of a target nucleic acid, wherein the request for collaborative development includes feature information about the target nucleic acid to be detected using the reagent; allowing a developer terminal to access a technology provider's development tool kit, wherein the development tool kit includes a performance test tool for the oligonucleotide to be included in the reagent, the performance test tool including one or more of software, a device, and instructional information; receiving from the developer terminal a result obtained from using the development tool kit, wherein the result includes performance data for the oligonucleotide; allowing the received result to be reviewed by the technology provider terminal or by a monitoring module of a collaborative development system; and allowing the developer terminal to access the review product, such that an oligonucleotide to be included in the reagent is selected.

In another aspect of the disclosure, there is provided a non-transitory computer-readable storage medium storing instructions executed by one or more processors for collaborative development of a reagent for detection of the target nucleic acid in a computing device, wherein when executed by the one or more processors, the instructions enable the computing device to perform: computer-implemented method for collaborative development of a reagent for detection of a target nucleic acid by a collaborative development system, comprising: receiving from a requester terminal a request for collaborative development of a reagent containing an oligonucleotide for detection of a target nucleic acid, wherein the request for collaborative development includes feature information about the target nucleic acid to be detected using the reagent; allowing a developer terminal to access a technology provider's development tool kit, wherein the development tool kit includes a performance test tool for the oligonucleotide to be included in the reagent, the performance test tool including one or more of software, a device, and instructional information; receiving from the developer terminal a result obtained from using the development tool kit, wherein the result includes performance data for the oligonucleotide; allowing the received result to be reviewed by the technology provider terminal or by a monitoring module of a collaborative development system; and allowing the developer terminal to access the review product, such that an oligonucleotide to be included in the reagent is selected.

In still another aspect of the disclosure, there is provided a computer-implemented method for collaborative development of a reagent for detection of a target nucleic acid by a collaborative development system comprising: receiving collaborative development application information including feature information about a target nucleic acid; determining a disease region where a disease associated with the target nucleic acid breaks out using the received feature information about the target nucleic acid; selecting a developer located in the disease region; and receiving from the developer terminal a result of performing a test for developing the reagent for detection of the target nucleic acid by the selected developer via a technology provider's support.

### Advantageous Effects of Invention

The features and advantages of the disclosure may be summarized as follows:

(1) According to the disclosure, the method, system, and storage medium automatically match the developer and technology provider suitable for the characteristics of collaborative development in response to a request for collaborative development, thereby increasing the efficiency of collaborative development.

(2) According to the disclosure, the method, system, and storage medium allow for simultaneous and regional restriction-free collaborative development between technology provider and developer over a network (that is, technology providers and developers perform multiple collaborative development projects), thereby enabling development of various reagents within a short time.

(3) According to the disclosure, the method, system, and storage medium allow technology providers, who are capable of developing reagent for detection of the target nucleic acids but do not have a room for complicated and time-consuming tests, to provide development techniques to developers, who can afford to conduct tests, and to direct them for development tests, thereby enabling quick development of various reagent for detection of the target nucleic acids.

(4) According to the disclosure, the method, system, and storage medium allow technology providers, who are capable of developing reagent for detection of the target nucleic acids but have difficulty in acquiring clinical samples for testing the performance of reagents, to provide development techniques to developers, who may easily obtain clinical samples, and to direct them for performance tests to develop various desired reagents.

(5) According to the disclosure, the method, system, and storage medium allow developers, who can afford to do tests for developing a reagent for detection of the target nucleic acid but lack development capability, to receive reagent development methods and know-how by collaborative development with technology providers.

(6) According to the disclosure, the method, system, and storage medium allow developers, who may easily acquire clinical samples for testing the performance of reagent for detection of the target nucleic acids, to develop their desired reagents by providing clinical samples to technology providers.

(7) According to the disclosure, the method, system, and storage medium allow technology providers, who are unable to acquire clinical samples in a disease outbreak region, to provide technical know-how to developers who are located where clinical samples may be acquired.

(8) According to the disclosure, the method, system, and storage medium allow multiple technology providers and developers to simultaneously proceed with collaborative development of a reagent for detection of the target nucleic acid capable of diagnosing various diseases breaking out in various regions. Thus, the reagent for detection of the target nucleic acid capable of detecting a number of diseases may be developed in the disease outbreak region within a short time and be used for the patients in the disease outbreak region.

(9) According to the disclosure, the method, system, and storage medium allows developers, who are located in some high-risk disease regions and who were incapable of properly developing a reagent for detection of the target nucleic acid for some diseases breaking out in the regions, to receive technical know-how from technology providers through collaborative development and develop necessary reagent for detection of the target nucleic acids.

(10) According to the disclosure, the method, system, and storage medium allow developers located in the disease outbreak region to receive technical know-how from technology providers, and test and develop a disease detection reagent which would otherwise be unwilling for development. A few patients in the disease region may be diagnosed for diseases using the developed reagent for detection of the target nucleic acid.

(11) In the case where an infectious disease spreads from the initial outbreak region to another, development of a reagent for detection of the target nucleic acid for diagnosis of the disease requires the clinical sample for each of the initial outbreak region and the second infection region due to possible mutation of the disease. The method, system, and storage medium allow technology providers to provide their know-how to the developers in the initial outbreak region and the second infection region via collaborative development and allow them to do tests for developing a reagent for detection of the target nucleic acid. Thus, a reagent for detection of the target nucleic acid capable of diagnosing the disease may be developed in both the regions at the same time.

(12) Development of reagent for detection of the target nucleic acids for detecting hereditary diseases which break out only in specific regions is delayed or impossible due to technology providers' difficulty in acquiring clinical samples. However, according to the disclosure, the method, system, and storage medium allow developers located in the disease region to easily develop a reagent for detection of the target nucleic acid using technology providers' know-how via collaborative development for doing tests using clinical samples for the hereditary disease.

(13) According to the disclosure, the reagent for detection of the target nucleic acid developed by collaborative development between developers in the disease region and technology providers providing technical know-how is sold and used in the disease region, allowing relevant enterprises in the disease region profits while allowing diagnosis organizations to reduce diagnosis costs for the developed reagent for detection of the target nucleic acid.

(14) According to the disclosure, the result of development test obtained by the developer and the review product by the technology provider are shared therebetween via the collaborative development system, thereby improving the efficiency of collaborative development.

(15) According to the disclosure, the method, system, and storage medium are useful particularly for development of a reagent for detection of multiple target nucleic acids that consumes much time and effort.

### BRIEF DESCRIPTION OF DRAWINGS

FIG. 1 is a view schematically illustrating an example of connecting a collaborative development system, a requester terminal, a developer terminal, and/or a development device, a technology provider terminal and, optionally, an evaluator terminal over a network according to the disclosure;

FIG. 2 is a view schematically illustrating a configuration of a collaborative development server according to the disclosure;

FIG. 3 is a flowchart illustrating a collaborative development method according to an embodiment of the disclosure;

FIG. 4 is a flowchart illustrating a collaborative development method according to an embodiment of the disclosure; and

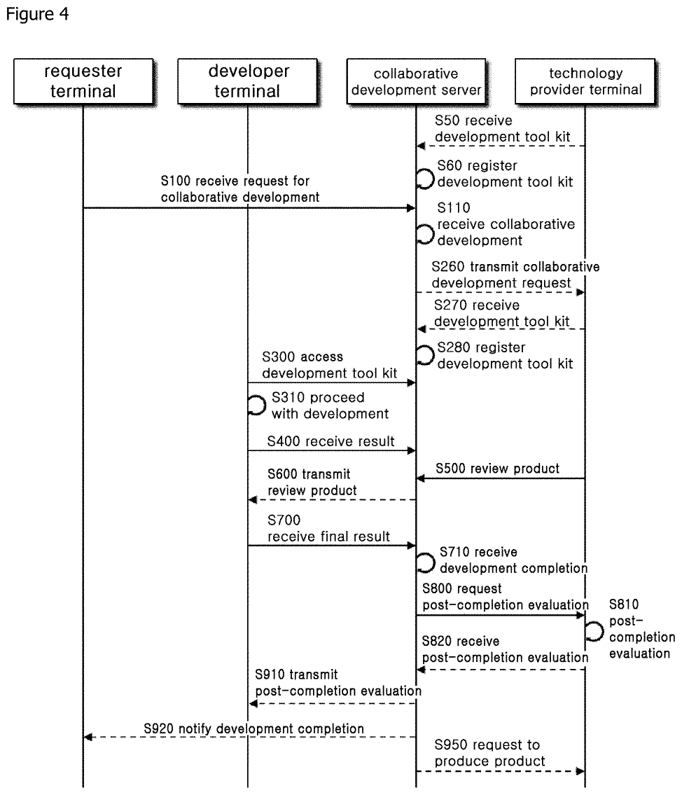

FIG. 5 is a view illustrating a configuration of accessing a development tool kit stored in a collaborative development server by a developer terminal or development device according to the disclosure.

### MODE FOR THE INVENTION

The configuration and effects of the disclosure are now described in further detail in connection with embodiments thereof. The embodiments are provided merely to specifically describe the disclosure, and it is obvious to one of ordinary skill in the art that the scope of the disclosure is not limited to the embodiments.

The inventors have endeavored to come up with a method or system for developing various reagent for detection of the target nucleic acids in a more efficient and simultaneous manner. As a result, the inventors have found that various reagents for detection of the target nucleic acids may be simultaneously developed regardless of places via collaboration between a technology provider, who provides technology, and a developer, who performs tests.

I. Configuration of Collaborative Development System According to the Disclosure

According to the disclosure, a collaborative development system 1000 includes a collaborative development server 100 which is connected with, e.g., terminals and development devices attending collaborative development, via a network.

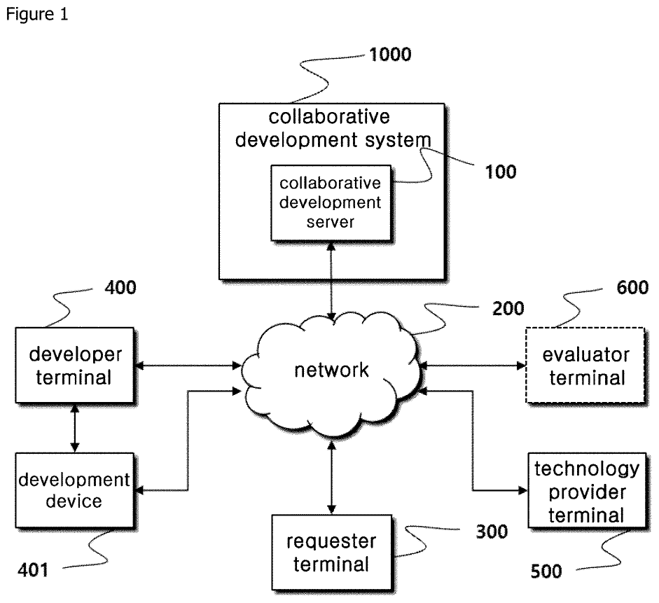

An example of connecting a collaborative development system, a requester terminal, a developer terminal, and/or a development device, a technology provider terminal and, optionally, an evaluator terminal over a network is illustrated in FIG. 1. Referring to FIG. 1, the collaborative development system 1000 according to the disclosure receives/transmits inputs/outputs necessary for collaborative development from/to each terminal, thereby facilitating collaborative development via each terminal.

The collaborative development server 100 of the collaborative development system 1000 is connected with a requester terminal 300, a developer terminal 400, and, a technology provider terminal 500 via a network. As necessary, the collaborative development server 100 is connected with an evaluator terminal 600 via the network.

The collaborative development server 100 in the collaborative development system 1000 according to the disclosure may receive a request for collaborative development according to the following implementation.

i) According to an embodiment, the collaborative development server 100 may receive a request for collaborative development from the requester terminal 300.

ii) According to another embodiment, the collaborative development server 100 may receive a request for collaborative development from the developer terminal 400. In this case, the developer terminal 400 corresponds to the requester terminal 300.

iii) According to still another embodiment, the collaborative development server 100 may receive a request for collaborative development from the technology provider terminal 500. In this case, the technology provider terminal 500 corresponds to the requester terminal 300.

The collaborative development server 100 may enable the developer terminal 400 for collaborative development to access a development tool kit of the technology provider for collaborative development.

According to an embodiment, a device may be included in the technology provider's development tool kit and be provided to the developer. According to another embodiment of the disclosure, the developer's device may be provided to the developer by connecting to the developer terminal 400.

The collaborative development server 100 may receive a result obtained from using the development tool kit from the developer terminal 400. Or, it may be received from the development device 401 connected with the developer terminal 400. The collaborative development server 100 enables a monitoring module 137 in the collaborative development server 100 or the technology provider terminal 500 to review the received result. The technology provider terminal 500 or the monitoring module 137 may generate a review product, and the collaborative development server 100 enables the developer terminal 400 to access the generated review product.

According to the disclosure, the receiving of the result from the developer terminal 400 and the reviewing the result by the monitoring module 137 of the collaborative development system 1000 or the technology provider terminal 500 are repeatedly performed for each test.

Thus, the review product, which is generated as the result is reviewed by the monitoring module 137 of the collaborative development system 1000 or the technology provider terminal 500, includes an indication of reperforming or approval of the performance test.

One or more developers may participate in collaborative development. Any one or more developers selected from a plurality of candidate developers desiring to attend collaborative development may participate in collaborative development. Thus, the developer terminal 400 is the terminal of a developer selected to perform collaborative development from the plurality of candidate developers. According to the disclosure, the collaborative development system 1000 may select a developer to obtain a clinical sample used for a test of collaborative development according to the following implementation example.

According to an embodiment, the selected developer terminal 400 is a terminal of a developer who has obtained or is able to obtain a clinical sample containing, or suspected of containing a target nucleic acid.

According to another embodiment, the selected developer terminal 400 is a terminal of a developer who is located in region where a disease associated with the target nucleic acid has broken out.

According to another embodiment, the selected developer terminal 400 is a terminal of a developer who is located in a region where a disease associated with a target nucleic acid breaks out or has broken out and who has obtained, or is able to obtain, a clinical sample from a subject having or suspected of having the disease.

According to another embodiment, the selected developer terminal 400 is a terminal of a developer who is in a region where a subject having or suspected of having a disease associated with a target nucleic acid is located.

According to another embodiment, the selected developer terminal 400 is a terminal of a developer who has obtained, or is able to obtain, a clinical sample from a subject having or suspected of having the disease associated with the target nucleic acid in the region where the disease breaks out and/or the region where the disease has broken out for a predetermined period.

Thus, the result transmitted from the developer terminal 400 may include a result of test using the clinical sample obtained by the developer.

According to the disclosure, the collaborative development system 1000 may be configured to implement a plurality of collaborative developments.

The collaborative development system 1000 according to the disclosure may perform evaluation of the feasibility of the collaborative development in response to a request for collaborative development.

In an example, the collaborative development server 100 may request for evaluation of the feasibility of the collaborative development by providing the received request for collaborative development to the evaluator terminal 600. The evaluator terminal 600 may perform evaluation of the feasibility of the collaborative development in response to a request for evaluation and provides the evaluation result of the feasibility of the collaborative development to the collaborative development server 100. The collaborative development server 100 initiates collaborative development when the evaluation result of the feasibility of collaborative development received from the evaluator terminal 600 is to approve the collaborative development.

In another example, the collaborative development server 100 may enable an evaluation management module 122 in the collaborative development server 100 to perform evaluation of the feasibility of the collaborative development in response to the request for collaborative development received from the requester terminal 300, the developer terminal 400, or the technology provider terminal 500. The evaluation management module 122 performs evaluation in response to the request for collaborative development to yield an evaluation result of the feasibility of collaborative development. The collaborative development server 100 initiates collaborative development when the evaluation management module 122 yields an result of approval.

In an example, the evaluator terminal 600 may include the technology provider terminal 500. In other words, the technology provider may perform evaluation of the feasibility of the collaborative development in response to the request for collaborative development via the technology provider terminal 500, generate an evaluation result of the feasibility of the collaborative development, and provide the result to the collaborative development server 100.

In response to the approval of collaborative development, the collaborative development server 100 may transmit a request for attending collaborative development to the developer terminal 400 and/or the technology provider terminal 500 and/or receive collaborative development attending information.

According to an embodiment, when a request for collaborative development is received from the requester terminal 300 and collaborative development is approved, the collaborative development server 100 may transmit a collaborative development attending request to one or more developer terminals 400 and/or technology provider terminals 500.

In response to the collaborative development attending request, the collaborative development server 100 may receive information indicating whether to attend collaborative development from the developer terminal 400 and/or the technology provider terminal 500 and select the developer and/or technology provider depending on the received information indicating whether to attend collaborative development.

The collaborative development system 1000 may derive a feature keyword for specifying a disease using feature information about the target nucleic acid contained in the received request for collaborative development and determine a disease region where the disease breaks out and/or has broken out, based on the derived feature keyword.

Thereafter, the collaborative development system 1000 may examine whether each developer is located in the determined disease region using location information from a plurality of developer terminals who desire to attend collaborative development and select the developer located in the disease region. One developer who is located in the disease region may be selected or, as necessary, a plurality of developers located in the disease region may be selected.

The collaborative development system 1000 may provide the evaluator terminal 600 with the information received from a plurality of developer terminals who desire to attend collaborative development, thereby sending a collaborative development attending request. The collaborative development system 1000 receives an evaluation result in response to the collaborative development attending request and selects a developer.

The collaborative development system 1000 provides a platform where collaborative development of a reagent for detection of the target nucleic acid may be performed by collaboration between the technology provider terminal 500 and the selected developer terminal 400 to perform the disclosed collaborative development. As necessary, the developer terminal 400 may include the development device 401 in performing collaborative development.

The collaborative development system 1000 may receive information about whether a clinical sample containing, or suspected of containing the target nucleic acid is obtained. The information may be received from the developer terminal 400 which sends the request for collaborative development, or the developer terminal 400 which desires to attend collaborative development. According to an embodiment, the information about whether the clinical sample is obtained may be included in the request for collaborative development or information transmitted for attending the collaborative development. According to an embodiment, the information about whether the clinical sample is obtained may be received from the developer terminal 400 before the collaborative development system 1000 selects the developer for performing collaborative development.

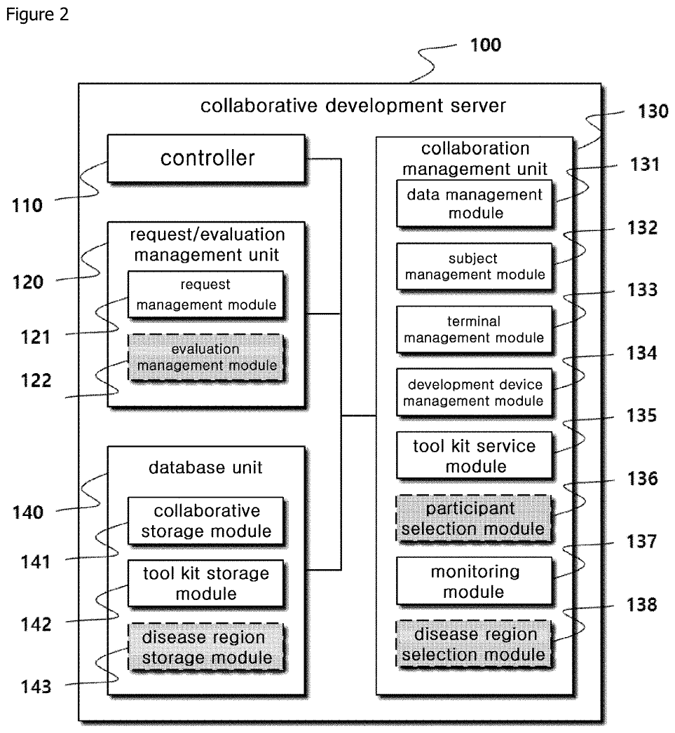

FIG. 2 is a view schematically illustrating a configuration of a collaborative development server according to the disclosure. Referring to FIG. 2, the collaborative development server 100 included in the collaborative development system 1000 includes the following components.

The collaborative development server 100 includes a controller 110 for controlling each component, a request/evaluation management unit 120 for managing a request or evaluation for collaborative development, a collaborative management unit 130 for substantially managing collaborative development, and a database unit 140 for storing test data and results obtained via collaborative development.

The controller 110 controls each component of the collaborative development server 100, used for collaborative development of a reagent for detecting multiple target nucleic acids. The control of each component by the controller 110 is described in further detail, along with the components of the server according to the disclosure.

The request/evaluation management unit 120 includes a request management module 121 and an evaluation management module 122.

The request management module 121 manages the start of a request for collaborative development and the steps before collaborative development evaluation. The request management module 121 performs proposal management that receives a request for collaborative development from a requester (or developer) and stores information according to the received request for collaborative development in a collaborative storage module 141 of the database unit 140. The request management module 121 may assign management numbers for the requester's request for collaborative developments. The management numbers may be sequentially assigned according to the requester's request for collaborative developments.

The request management module 121 may receive a request for collaborative development from the requester terminal as in the following embodiment.

According to an embodiment, the request management module 121 may use a webpage scheme for request for collaborative developments and allow the requester terminal to access the webpage and enter the content for collaborative development.

According to another embodiment, the request management module 121 may provide a file in which the content for request for collaborative development may be created and receive the content for collaborative development, which the requester has entered to the requester terminal in the form of a file.

The request management module 121 receives a request for collaborative development using a scheme selected from among request for collaborative development schemes.

The request management module 121 reviews the collaborative development content contained in the received request for collaborative development and determines whether there is an error. The review may include a preliminary evaluation for performing a formal review on the request for collaborative development and a substantial evaluation for performing a review on the merits of the scope and field of the collaborative development. Or, the evaluation management module 122 may perform the preliminary evaluation and substantial evaluation both.

During the preliminary evaluation or substantial evaluation, it is also reviewed whether a developer for substantially performing collaborative development has been chosen. The request may be in charge of substantial development in collaborative development and may request a separate developer who performs substantial development.

Where the request management module 121 performs a preliminary evaluation, the request management module 121 provides the result of the preliminary evaluation on the request for collaborative development to the controller 110.

The controller 110 reviews the received preliminary evaluation on the request for collaborative development and, if there is no error, controls the evaluation management module 122 to perform the substantial evaluation. The results of preliminary evaluation may be derived as follows.

i) Where No Error Occurs with the Result of Review on the Request for Collaborative Development,

The controller 110 sends a request for substantial evaluation on collaborative development to the evaluation management module 122 to be able to perform a substantial evaluation on the request for collaborative development.

ii) Where an Error Occurs with the Result of Review on the Request for Collaborative Development,

The controller 110 sends a request for supplementing the request for collaborative development to the request management module 121. In response thereto, the request management module 121 requests the requester terminal to supplementing the request for collaborative development. The request management module 121 receives the supplemented request for collaborative development and performs the preliminary evaluation again.

The evaluation management module 122 performs evaluation in two steps of collaborative development.

The first step is a request evaluation step according to a request for collaborative development, and the second step is a post-completion evaluation step according to the result of collaborative development. In each evaluation step, the evaluation management module 122 operates as follows.

1) Evaluation Management Module in Request Evaluation Step

Upon receiving a request for collaborative development, the controller 110 controls the evaluation management module 122 to perform an evaluation on the feasibility of collaborative development in response to the request for collaborative development.

The evaluation management module 122 selects an evaluator when the controller 110 requests an evaluation of collaborative development. The evaluator should be able to evaluate the feasibility of collaborative development using the request for collaborative development.

The evaluation management module 122 may select one or more evaluators from a plurality of candidate evaluators registered in the collaborative development system 1000. A technology provider attending the collaborative development may be among the evaluators selected by the evaluation management module 122. The evaluation management module 122 may select only the technology provider attending the collaborative development as the evaluator.

The evaluation management module 122 may select evaluation items of evaluation criteria to be provided to the evaluator depending on the field of collaborative development. The evaluation management module 122 provides a development evaluation request including the selected evaluation items to the evaluator terminal. The evaluation items may be ones previously determined or may be evaluation items according to the results trained based on artificial intelligence (AI) using deep learning or machine learning.

According to an embodiment, the evaluation management module 122 may use a webpage scheme for the request for evaluation of the feasibility of collaborative development and allow the evaluator terminal to access the webpage and enter the result of evaluation on each evaluation item.

According to another embodiment, the evaluation management module 122 may provide a file including evaluation items to the evaluator terminal, and the evaluator may receive the results of evaluation entered through the evaluator terminal in the form of a file and perform an evaluation on the collaborative development.

The evaluation management module 122 determines the evaluator's results of evaluation received using a scheme selected from among evaluation request schemes and provides the result of determination of the evaluation to the controller 110.

The request evaluation performed by the evaluation management module 122 may be performed one or multiple times.

The controller 110 provides an indication of approval or rejection for the collaborative development to the requester terminal in response to the request for collaborative development, depending on the result of determination of the evaluation.

If the evaluation determination result is an indication of rejection for the collaborative development, the controller 110 may instruct to supplement the request for collaborative development or stop proceeding with the request for collaborative development. In contrast, if the evaluation determination result is an indication of approval for collaborative development, the controller 110 performs a next process for the request for collaborative development.

2) Evaluation Management Module in Post-Completion Evaluation Step

Upon receiving the final result for development from the developer terminal (or development device) which has performed collaborative development, the evaluation management module 122 may perform a post-completion evaluation on the feasibility of producing the reagent using the final result. The post-completion evaluation is performed in the same manner as the request evaluation on the request for collaborative development.

In other words, the evaluation management module 122 may select an evaluator who may perform a post-completion evaluation, send a request for post-completion evaluation to the selected evaluator, receive the result of evaluation from the evaluator terminal of the selected evaluator, and determine the feasibility of reagent production for the completed collaborative development.

According to an embodiment, the evaluator selected in the post-completion evaluation step by the evaluation management module 122 may be identical to the evaluator selected in the request evaluation step. This is for the feasibility for reagent production to be evaluated by the evaluators who have evaluated the feasibility of collaborative development in the request evaluation step.

According to another embodiment, the evaluator selected in the post-completion evaluation step by the evaluation management module 122 may differ from the evaluator selected in the request evaluation step.

Evaluations performed by the evaluation management module 122 may include a request evaluation for request for collaborative development and a post-completion evaluation for the results of collaborative development as described above. The collaborative development system 1000 may perform the request evaluation with the help of the evaluation management module 122 and perform post-completion evaluation with the help of the selected evaluator. Alternatively, the collaborative development system 1000 may perform both evaluations with the help of the evaluation management module 122.

When the request evaluation and/or post-completion evaluation is needed, the evaluation management module 122 performs the evaluation using the evaluation criteria stored (or operated) in the evaluation management module 122.

The evaluation criteria may be configured to be able to evaluate each of the request for collaborative development part and/or final result part, and evaluation may be performed per technical field of collaborative development.

The evaluation management module 122 may be operated as in the following implementation examples.

i) The evaluation management module 122 may perform each of the request evaluation and the post-completion evaluation with the help of different evaluators.

ii) The evaluation management module 122 may perform one of the request evaluation and the post-completion evaluation with the help of the evaluation management module 122 and perform the other of the request evaluation and the post-completion evaluation with the help of an evaluator.

iii) The evaluation management module 122 may perform only one of the request evaluation and the post-completion evaluation with the help of any one of the evaluation management module 122 and an evaluator.

iv) The evaluation management module 122 may omit both the request evaluation and the post-completion evaluation.

v) The collaborative development server 100 may not include the evaluation management module 122 and, thus, may not perform both the request evaluation and the post-completion evaluation.

As described above, the collaborative development server 100 may operate the evaluation management module 122 according to any one selected from the five implementation examples. The selection may be made depending on technical fields and/or types of collaborative development.

If collaborative development is approved according to the evaluation of the feasibility of collaborative development, a process for collaborative development is performed via the collaborative management unit 130.

The collaborative management unit 130 includes a data management module 131, a subject management module 132, a terminal management module 133, a development device management module 134, a tool kit service module 135, a participant selection module 136, a monitoring module 137, and a disease region selection module 138.

According to an embodiment, the collaborative management unit 130 may not include the participant selection module 136.

According to another embodiment, the collaborative management unit 130 may not include the disease region selection module 138.

The data management module 131 manages the data mutually provided among the technology provider terminal, collaborative development system 1000, and the developer terminal.

The developer terminal (including the development device) uploads the test results related to the collaborative development performed by the developer on the developer terminal and/or development device to the collaborative development system 1000. The data management module 131 identifies the developer's test result uploaded to the collaborative development system 1000 using the unique identifier assigned to the developer and, corresponding thereto, stores it in the collaborative development storage module of the database unit 140.

When the technology provider requests to view the test results stored according to the collaborative development, for reviewing purposes, the data management module 131 authenticates the technology provider and test results using the unique identifier assigned to the technology provider. The data management module 131 may provide the test results to the authenticated technology provider terminal. The provided test results may be provided in such a manner that the technology provider terminal may download the result file or may be provided via a viewer where copying, downloading, or screen capturing for the test result is unavailable.

The data management module 131 receives the review product, which is feedback data provided by the monitoring module 137 and/or the technology provider according to the reviewing of the test result, and stores it in the collaborative storage module 141 of the database unit 140. The data management module 131 allows the developer to view the review product (feedback data).

As necessary, the data management module 131 may cipher and store various data provided from the developer terminal (including the development device) and/or the technology provider terminal. The various data provided to the collaborative development system 1000 may be encrypted by the developer terminal (including the development device) and/or the technology provider terminal and be provided to the collaborative development system 1000. The data management module 131 may perform additional encryption on the data previously encrypted.

The data management module 131 may encrypt various data using the user identifier assigned to the corresponding collaborative development. Thus, the developer and technology provider may view the various data stored in the collaborative development storage module of the database unit 140 using their respective user identifiers on their respective terminals. The user identifier assigned to each may serve as a decryption key for use in viewing.

The encryption and decryption may be performed by the data management module 131 preferably using the user identifier assigned to each but may also be carried out using various security solutions, such as an ID/PW scheme or passcode scheme.

The subject management module 132 performs, e.g., generation of a subject number for the collaborative development, generation and discard (terminate) of the user identifier, and notification of progress/termination of collaborative development according to the development process from the start to end of the collaborative development. Specifically, the subject management module 132 performs the following in each process of collaborative development.

i) Generation of Subject Number

Where collaborative development is approved in the request evaluation (evaluation of the feasibility of collaborative development), the subject management module 132 generates a subject number for the collaborative development. The subject number may be generated to be able to identify the technical field and scope of development for the collaborative development. Additionally, the subject number may be generated in consideration of the subject period, and may include, but is not limited to, letters and numbers.

ii) Generation and Discard of User Identifier

After a subject number is generated and a developer and a technology provider are selected, the subject management module 132 provides the developer and technology provider with user identifiers for identifying the collaborative development. The user identifiers may be used for identifying the developer and technology provider, use authentication for use in, and access to, the development device or development tool kit used for collaborative development, and encryption of the data (including documents) generated by the developer and technology provider.

Thus, the user identifier includes, e.g., user IDs and passwords which match the subject number to be able to identify the developer and the technology provider. The user identifier includes, e.g., a certificate for access to, and approval of use of, the development device or the development tool kit. The user identifier includes an encryption key used for data encryption. The user identifier includes a decryption key by which the developer or technology provider attending the collaborative development may view the encrypted data.

When the collaborative development is terminated, the subject management module 132 discards or stops use of the user identifiers individually provided to the developer and the technology provider. As necessary, even after the collaborative development is terminated, some functions of the user identifier may be maintained to be able to view the data generated during the collaborative development.

The user identifier generated by the subject management module 132 has a predetermined development period during which collaborative development is carried out. Thus, the user identifier may be activated during the development period, but may automatically be deactivated after the development period.

iii) Notification of Progress/Termination of Collaborative Development

Where the progress of collaborative development is approved in the request evaluation (evaluation of the feasibility of collaborative development), the subject management module 132 provides a notification of starting the subject, including the generated subject number and user identifiers, to the developer terminal and the technology provider terminal.

If the subject management module 132 receives the final result for collaborative development and the post-completion evaluation is completed, the subject management module 132 provides a notification of the termination (completion) of collaborative development to the developer terminal and technology provider terminal.

The notification of the termination (completion) of collaborative development may include data for stopping the use of the user identifiers. Where the user identifiers provided to the developer and technology provider are configured to be automatically stopped from use after the collaborative development period, the collaborative development termination (completion) notification may not include the data for stopping the use of the user identifiers.

The terminal management module 133 controls (e.g., approves/rejects) the access to the collaborative development system 1000 by the developer terminal and technology provider terminal and manages a log therefor. Where a plurality of collaborative developments are in progress and multiple developer terminals and multiple technology provider terminals request a connection to the collaborative development system 1000, the terminal management module 133 identifies each terminal and establishes or stops a session. Each terminal in session may use the tool kit service module 135, upload the data obtained during the collaborative development, and/or view or download the data stored in the database unit 140, using the provided user identifier.

It is preferable to allow a session only for terminals authenticated via their user identifiers.

The development device management module 134 manages the development device used for collaborative development. As the collaborative development is in progress, the developer registers its own device and/or the device provided from the technology provider, as a device used for collaborative development, and the development device management module 134 monitors the condition of the development device.

For example, the development device management module 134 determines whether firmware suitable for the development device has been installed or updated.

The development device management module 134 determines whether the development device may sufficiently perform a test according to collaborative development. For example, the development device management module 134 may determine whether a nucleic acid amplifying device (e.g., a PCR device) is capable of a real-time nucleic acid amplification test or whether the number of wells in a test plate meets a standard needed for the collaborative development.

Further, the development device management module 134 receives an error report for the development device registered for collaborative development and provides the history or log to the developer terminal and/or technology provider terminal. Thereafter, the developer and/or technology provider transmits a solution to the error to the developer terminal or the development device, which has caused the error, via the collaborative development system 1000.

As a representative example, nucleic acid amplifying devices may be connected to the developer terminal. The nucleic acid amplifying devices are devices necessary for developing a multiplex reagent for detection of the target nucleic acid and, particularly, include real-time nucleic acid amplifying devices, e.g., real-time PCR devices.

The tool kit service module 135 serves the development tool kit provided by the technology provider to the developer terminal and/or development device in a cloud type or software-as-a-service (Saas) type.

The tool kit may be in the form of a package and be downloaded, installed and/or used on the developer terminal and/or development device. However, as necessary, the tool kit, although used by the developer terminal and/or development device, may not be downloaded but may be used by access to the collaborative development system 1000.

In this case, the collaborative development system 1000 allows the developer terminal and/or development device to use the tool kit in an online service type (e.g., cloud type) using the tool kit service module 135.

When a technology provider is determined as the request for collaborative development is approved, the controller 110 registers the tool kit of the determined technology provider in the tool kit service module 135 to be used by the developer in a cloud type and/or SaaS type.

The tool kit service module 135 of the collaborative development server 100 may serve, online, the development tool kit provided to the developer terminal and/or development device.

The tool kit service module 135 may receive the data used for a tool test while serving the tool, online, to the developer terminal and/or development device, as follows.

i) The data received to perform a test in the tool included in the tool kit service module 135 may be received from the developer terminal and/or development device.

In this case, the data received from the developer terminal and/or development device may be stored in the collaborative storage module 141 of the database unit 140 by the data management module 131 and be provided to the tool of the tool kit service module 135. Or, the data received from the developer terminal and/or development device may be temporarily stored in the tool kit service module 135 by the data management module 131 and be provided to the tool of the tool kit service module 135.

ii) The data received to perform a test in the tool included in the tool kit service module 135 may be the result of a test performed in other tool of the tool kit service module 135.

In this case, the result of the test performed in the other tool may be stored in the collaborative storage module 141 of the database unit 140 by the data management module 131 and be provided to the tool of the tool kit service module 135. Or, the result of the test performed in the other tool may be temporarily stored in the tool kit service module 135 and be provided to the tool of the tool kit service module 135.

Meanwhile, the data (result) received to perform a test in the tool of the tool kit service module 135 is the result of the test performed in the tool kit used for collaborative development.

Further, the tool kit service module 135 may provide the data generated as any one tool performs a test, as follows.

i) The generated data may be used as input data for other tool of the tool kit service module 135.

ii) The generated data may be provided to the developer terminal and/or development device performing the corresponding collaborative development.

iii) The generated data may be provided to the monitoring module 137 in the collaborative development server 100 to be verified by the collaborative development system 1000 and/or the technology provider.

The tool kit service module 135 may receive support for encryption for the incoming data and decryption for the outgoing data.

i) Where the data received for use in the tool is encrypted and received, the tool kit service module 135 may receive decryption of the encrypted data from the collaborative development system 1000.

ii) Where the test data generated as a test is performed in the tool, the tool kit service module 135 may receive encryption of the test data from the collaborative development system 1000.

The encryption or decryption may be performed by the controller 110 in any one or more of the data management module 131, subject management module 132, terminal management module 133, development device management module 134, or tool kit service module 135 included in the collaborative development server 100.

Collaborative development is performed by a plurality of developers according to a plurality of collaborative developments, and the tool kit service module 135 may receive requests for use of the tool from the plurality of developer terminals and/or development devices. Each tool of the tool kit service module 135 may sequentially process the requests for use of the tool from the plurality of developer terminals and/or development devices and approve the use of the tool.

Where any one tool provided by the tool kit service module 135 is used in different collaborative developments, the tool kit service module 135 may automatically change, e.g., parameters or other setting values, used for the tool to be suited for the collaborative development performed by the developer terminal and/or development device requesting to use the tool. In this case, for the automated change of the setting values, the controller 110 may recognize the user identifier of the developer terminal and/or development device which tries to access for use of the tool, and the tool kit service module 135 may make a change to be suited for the collaborative development corresponding to the user identifier recognized by the controller 110.

According to an embodiment, the tool kit service module 135 may be included in the collaborative development server 100 to provide a service to the developer.

According to another embodiment, the tool kit service module 135 may not be included in the collaborative development server 100 but may provide a service in a separate position. The tool kits provided from a plurality of technology providers each may include one or more tools. If each tool uses many resources, it may be configured as a separate tool kit service server independent from the collaborative development server 100 and provide a service to the developer terminal and/or development device.

According to an embodiment, the development tool kit provided from the technology provider to the developer includes tools necessary to develop a reagent for detection of the target nucleic acid.

The tools may include an oligonucleotide design tool, an oligonucleotide performance test tool, a performance test analysis tool, a performance test documentation tool, an encryption supporting tool, and/or other development tools. The technology provider may have at least one or more tools of those listed above and, as collaborative development proceeds, the technology provider may selectively provide its own tools to the developer.

Among the tools, the tool provided in the form of software (including instructional information) may be transmitted to the collaborative development system 1000 and/or developer terminal via the technology provider terminal. In this case:

i) The tool may be in the form of an install file that may be installed on the developer terminal and/or development device; or

ii) The tool may be in the form of an executable file that may be executed without installation.

The instructional information is in the form of a document, image, video, audio, or other file that may be visually/auditorily identified by the developer via the developer terminal and/or development device.

Each tool may include either or both of software or/and instructional information.

Each tool is described below in greater detail in the section for a method for collaborative development of a reagent for detection of the target nucleic acid according to the disclosure.

According to an embodiment, the participant selection module 136 selects a developer and/or technology provider who attends collaborative development when the collaborative development is performed by a requester. The participant selection module 136 may select the developer and technology provider using developer information and technology provider information stored in the collaborative storage module 141 of the database unit 140. The developer information and the technology provider information are described below in detail in connection with the collaborative storage module 141.

According to another embodiment, the participant selection module 136 selects a developer included in a disease region selected by the disease region selection module 138. Where a disease region where a disease corresponding to the reagent for detection of the target nucleic acid to be developed via collaborative development is selected, a developer may be selected from a plurality of candidate developers requesting to attend the collaborative development or requesting to apply for the collaborative development. The selection of a disease region is described below in detail in connection with the disease region selection module 138.

According to another embodiment, the participant selection module 136 selects a developer included in a disease region selected by the disease region selection module 138. A developer who has already obtained or is able to obtain a clinical sample containing, or suspected of containing the target nucleic acid to be developed via collaborative development may be selected from among a plurality of candidate developers requesting to attend the collaborative development or requesting to apply for the collaborative development.

The participant selection module 136 may select a developer and/or technology provider that attends collaborative development in each following case according to an instruction from the controller 110.

1) If You Choose a Developer,

If a request for collaborative development is received from the requester, and collaborative development proceeds, a developer for the collaborative development is selected. The developer may be one from various industrial sectors depending on the field of the collaborative development and/or the kind of reagent.

Among a plurality of candidate developers, a developer suitable for the collaborative development is selected. The selected developer is a developer previously registered in the collaborative development system 1000, and the technical field and/or the kind of reagent available for collaborative development may be registered in the collaborative development system 1000 so that such selection is possible. As necessary, a plurality of developers may be selected. A technology provider and an evaluator may be selected as necessary.

The participant selection module 136 provides a request for attending (development) collaborative development to the selected developer and receives information indicating whether the developer is to attend the collaborative development from the developer terminal. Upon receiving information indicating that the selected developer cannot attend collaborative development from the developer terminal, the participant selection module 136 selects other developer and provides a request for attending the collaborative development.

The participant selection module 136 may select at least any one or more developers from among a plurality of candidate developers applying for the collaborative development. In this case, the selected developer may be a developer included in the disease region or a developer who has already obtained or is able to obtain a clinical sample containing or suspected of containing the target nucleic acid. Among the developers who have applied for the collaborative development and have been primarily selected, a developer included in the disease region may be secondarily selected.

2) If the Requester and Developer are the Same,

Where the request is the same as the developer, the requester (developer) may attend and perform the collaborative development. In other words, the development field and/or kind of reagent included in the request for collaborative development is for the ones that may be developed by the requester (developer). Thus, developer selection may be omitted.

On the other hand, even when the requester is the developer, developer selection may be performed. For example, a developer who is able to obtain a “clinical sample” used for a test for developing a reagent for detection of the target nucleic acid via collaborative development may be selected. Thus, for a developer who has requested collaborative development, it is determined whether the developer is located in the disease region set according to the feature information for the target nucleic acid or whether the developer is able to obtain the clinical sample. Where the developer is located in the disease region or the developer is able to obtain the clinical sample, the collaborative development proceeds. Unless the requester (developer) who has requested collaborative development is located in the disease region or is able to obtain the clinical sample, the collaborative development system 1000 may i) select other developer for collaborative development or ii) cease the collaborative development.

Thus, the collaborative development system 1000 may further perform the step of determining whether the requester (developer) who has requested collaborative development is located in the disease region or she is able to obtain the clinical sample. This is for selecting a developer located in the region where a disease corresponding to the reagent for detection of the target nucleic acid to be developed via collaborative development breaks out or a developer who is capable of obtaining the clinical sample.

Even when the requester is the developer, an additional developer may be selected. For example, the requester (developer) may be in charge of a predetermined portion of the overall collaborative development, and the other selected developer may be in charge of the rest of the collaborative development. As necessary, a plurality of developers may be selected.

A technology provider and an evaluator may be selected as necessary.

The participant selection module 136 provides a request for attending (development) collaborative development to the selected additional developer and receives information indicating whether the developer is to attend the collaborative development from the developer terminal. Upon receiving information indicating that the selected developer cannot attend collaborative development from the developer terminal, the participant selection module 136 selects other developer and provides a request for attending the collaborative development.

When there is a request for collaborative development from the requester (developer), the participant selection module 136 may select at least any one or more additional developers from a plurality of candidate additional developers applying for the requested collaborative development. In this case, the selected additional developer may be a developer included in the disease region or a developer who may obtain the clinical sample. Among the developers who have applied for the collaborative development and have been primarily selected, a developer included in the disease region may be secondarily selected.

3) If the Technology Provider of the Plurality,

Among technology providers with various skills depending on the development field and/or kind of reagent according to collaborative development, a technology provider who has and is capable of providing development device and/or a tool kit suitable for the collaborative development to be performed is selected. In this case, the technology provider is a technology provider previously registered in the collaborative development system 1000. The development device and/or development tool kit of the technology provider selected by the technology provider selection may be provided to the developer terminal and/or technology provider terminal.

The participant selection module 136 provides a request for attending (development) collaborative development to the selected technology provider and receives information indicating whether the technology provider is to attend the collaborative development from the technology provider terminal. Upon receiving information indicating that the selected technology provider cannot attend collaborative development from the technology provider terminal, the participant selection module 136 selects other technology provider and provides a request for attending the collaborative development.

If the participant selection module 136 starts the participation of technology providers having and capable of providing the development device and/or tool kit suitable for the requested collaborative development, the participant selection module 136 may select at least any one or more additional technology providers among the plurality of additional technology providers applying for attending the collaborative development.

The monitoring module 137 reviews the result received from the developer terminal and/or development device. The monitoring module 137 may provide an authority to access the received result to the technology provider. The collaborative storage module 141 of the database unit 140 stores test results for the collaborative development, and each stored result is classified and stored by the collaborative development and a user identifier therefor. The monitoring module 137 may provide the user identifier-assigned result corresponding to the user identifier of the technology provider to be accessed by the technology provider.

Examples of access to the result may include a method in which the technology provider views the result stored in the database unit 140 via the technology provider terminal and a method in which the technology provider downloads the file of the result to be able to verify the result using the development tool kit provided in the technology provider terminal. According to another embodiment, a uniform resource locator (URL) by which access to the developer terminal may be gained may be provided to the technology provider under the control of the collaborative development system 1000, allowing the technology provider to directly view and/or download the result from the developer terminal. In this case, other methods than using the URL may be adopted to provide access to the developer terminal using a communication network.

The result provided from the monitoring module 137 to the technology provider may be encrypted, and the technology provider may identify the encrypted result using the user identifier or a separate decryption module.

The monitoring module 137 receives the review product from the technology provider terminal. The review product includes feedback information for the result provided to be viewed or downloaded by the technology provider terminal, and the review product includes information for verifying the result of the performance test and reperforming or approving a performance test thereon.

The monitoring module 137 stores the review product received from the technology provider terminal in the collaborative storage module 141 of the database unit 140 to be classified using the user identifier. Thereafter, the monitoring module 137 may provide an authority for the developer terminal to gain access to the stored review product. The review product includes the feedback information for the technology provider's result and, thus, requires the developer's confirmation. To that end, the monitoring module 137 provides the stored review product to the developer terminal for the developer to view or download, thereby allowing the developer to view or download the review product.

The monitoring module 137 uses the following methods to provide the received test result and/or review product.

A first method is an active method that, when the result is received, transmits the result to the technology provider terminal and/or the developer terminal.

A second method is a passive method that receives the result and, upon receipt of a request for the result from the technology provider terminal and/or the developer terminal, transmits the result.

The monitoring module 137 may verify the result received from the developer using a result verification technique provided from the technology provider and generate a review product therefor. The result verification technique is a technique that may train the monitoring module 137 with a number of test results and review products therefor to allow the monitoring module 137 itself to read the results and derive a result, such as approval or reperforming of a performance test.

The result verification technique may use artificial intelligence (AI)-based trained results using, e.g., deep learning or machine learning according to an embodiment.

Or, the monitoring module 137 may have a result verification technique, and the technology provider may provide a number of test results and review products therefor used for the result verification technique.

The disease region selection module 138 may specify a disease from feature information about the target nucleic acid in the request for collaborative development transmitted from the technology provider's, developer's, or requester's terminal to request collaborative development and may select a region in which the specified disease is breaking out and/or the specified disease has broken out within a predetermined period.

The feature information about the target nucleic acid in the request for collaborative development contains information for specifying the reagent for detection of the target nucleic acid to be developed via collaborative development.

The disease region selection module 138 extracts a feature keyword from the feature information about the target nucleic acid for specifying the disease that may be detected by the developed reagent. According to an embodiment, the extracted feature keyword is compared with information and the disease keywords stored, matched with their respective diseases stored in the disease region selection module 138 in the database unit 140, and a disease with a high matching rate is specified as the disease that may be detected by the reagent to be developed via collaborative development. According to another embodiment, the disease may be specified with the disease name contained in the feature information about the target nucleic acid. According to another embodiment, the disease may be specified with the disease classification symbol contained in the feature information about the target nucleic acid.

Thereafter, the disease region selection module 138 refers to the disease region storage module 143 in the database unit 140 to select the region, in which the specified disease is currently breaking out and/or the specified disease has broken out within a predetermined period, using the name of the specified disease.

The disease region storage module 143 stores disease names, disease features, disease keywords, or disease outbreak regions classified in various categories, such as disease, region, time of outbreak, first outbreak region, and secondary infection region.

The disease region information is described below in greater detail in connection with the disease region storage module 143.

Thus, the disease region selection module 138 may select the disease region which is the region where the specified disease is breaking out and/or the specified disease has broken out within a predetermined period by comparing the specified disease with the disease outbreak regions stored in the disease region storage module 143.

Thereafter, when the participant selection module 136 selects the developer, it may be determined whether the developer is located in the selected disease region, and a developer located in the disease region may be selected.

The reason for selecting a developer in the selected disease region is why it is easy to acquire a clinical sample in the region where the disease is breaking out. University, tertiary hospital, research centers, or enterprises located in the region which is not the disease outbreak region may also possess various clinical samples. However, the kind and amount of clinical samples they own may be limited and, for some diseases, they may have or secure no clinical samples.

Thus, it may be preferable to perform a test for a reagent for detection of the target nucleic acid by the developer located in the disease outbreak region.

The database unit 140 includes the collaborative storage module 141 for storing information used for collaborative development and the tool kit storage module 142 for storing the development tool kit provided from the technology provider. Although FIG. 2 illustrates that the database unit 140 is included in the collaborative development server 100 according to an embodiment, the database unit 140 may alternatively be positioned outside the collaborative development server 100.

Although FIG. 2 illustrates that the database unit 140 is included, as a single component, in the collaborative development server 100 according to an embodiment, the database unit 140 may alternatively be included, as multiple separate components, in the collaborative development server 1000.

Although FIG. 2 illustrates that the database unit 140 includes the collaborative storage module 141 and the development tool kit storage module 142 according to an embodiment, one or more collaborative storage modules 141 and development tool kit storage modules 142 may be included in each of a plurality of database units 140, according to another embodiment.

The collaborative storage module 141 stores information used for collaborative development in the following classifications.

(i) User Information

The collaborative storage module 141 stores user information about the developer, technology provider, requester, or evaluator. The users may be individuals, research centers, enterprises, or hospital. The collaborative storage module 141 stores information for determining whether each user may attend collaborative development.

For example, the collaborative storage module 141 may store, e.g., information for determining whether the developer is capable of performing various performance tests in the collaborative development, such as his major, development career, or history of participation in collaborative development.

The collaborative storage module 141 may include, e.g., information for determining the technology providable for collaborative development for the technology provider, development tool kit, development device, and/or the capability of producing the developed product.

The collaborative storage module 141 may include, e.g., information for comprehensively determining the feasibility of collaborative development, the developer's capability of collaborative development, and whether it is commercialized using the request for collaborative development received from the developer or requester for the evaluator.

The user information may be provided to the participant selection module 136 and be used for selecting a developer and/or technology provider. The user information may also be provided to the evaluators in the request evaluation step and be used for evaluation of the request for collaborative development.

(ii) Performance Test Data

The collaborative storage module 141 stores, e.g., the results of a performance test performed by the developer using the developer terminal and/or development device in the ongoing collaborative development. The collaborative development system 1000 may simultaneously perform a plurality of collaborative developments, and the collaborative storage module 141 receives the results of performance tests performed by a plurality of developers and stores them to be classified using user identifiers by the data management module 131.

(iii) Review Product

The collaborative storage module 141 reviews the performance test data received from the developer and stores the generated review product. The review product may be generated from the monitoring module 137 and/or the technology provider's technology provider terminal, and the collaborative storage module 141 stores the review product and stores it to be classified by the data management module 131 using the user identifier.

(iv) Big Data Used for Result Verification Technique

The collaborative storage module 141 stores data, such as myriad test results and review products therefor, used for the result verification technique of the monitoring module 137. The data is received from the technology provider terminal and is stored by the data management module 131. The ii) performance test data and/or iii) review product generated in the collaborative development may also be stored, as big data, by the data management module 131.

(v) Request for Collaborative Development and Evaluation Information

The collaborative storage module 141 stores information about the request for collaborative development received from the requester. The request for collaborative development information may be stored in association with the requester's user information.

The collaborative storage module 141 also stores the evaluation information about the evaluation performed by the evaluator and/or the evaluation management module 122. The evaluation information may be stored in association with the evaluator' user information.

The evaluation information may be classified into evaluation information in the request evaluation step and evaluation information in the post-completion evaluation step and, when each class of evaluation information is received, it is stored to be classified by the data management module 131 using the user identifier.

(vi) Log

The collaborative storage module 141 receives a log generated while using the development device or development tool kit (including the development tool kit service module 135) used by the developer and/or technology provider and stores it by the data management module 131. The log may include at least any one or more of the date, time, user, place, and/or number of times or such information for the use of the development device and/or development tool kit.

The above-enumerated information may be encrypted and stored by the data management module 131. The data provided to the terminal of each of the developer, technology provider, and evaluator may be decrypted and transmitted by the data management module 131.

The tool kit storage module 142 stores the development tool kit provided from the technology provider. The development tool kit is provided in the form of software and/or a file and is stored to be classified by the data management module 131 using the user identifier.

The tool kit may be provided from the technology provider (i) before the collaborative development is performed or (ii) after the collaborative development has been approved.

Thus, the development tool kit of each technology provider stored in the tool kit storage module 142 may be a development tool kit used for collaborative development or may be a development tool kit previously stored for collaborative development which is to be performed in the future.

The development tool kit may be provided from the technology provider or provided to the developer according to the following implementation examples.

1) Time when Development Tool Kit is Provided from Technology Provider

The collaborative development system 1000 may receive the development tool kit from the technology provider via the technology provider terminal according to the following implementation example.

i) The technology provider may provide the collaborative development system 1000 with its own development tool kit via the technology provider terminal while sending a request for registration to the collaborative development system 1000 for collaborative development.

ii) The technology provider may provide the collaborative development system 1000 with its own development tool kit via the technology provider terminal after selected to attend the collaborative development.

iii) The technology provider may provide its own development tool kit to the developer terminal and/or development device via the technology provider terminal after selected to attend the collaborative development.

2) Path of Providing Development Tool Kit

The development tool kit may be provided from the technology provider terminal to the collaborative development system 1000. The development tool kit may be provided by the collaborative development system 1000 to the developer terminal. The development tool kit may be provided from the technology provider terminal to the developer terminal. The development tool kit may be provided to the developer by the technology provider.

The development tool kit transmitted between the technology provider terminal, collaborative development system 1000, and/or developer terminal may be provided by the following various methods.

i) Providing Online

Among the development tool kits, the tool provided in the form of software (including instructional information) may be transmitted to the collaborative development system 1000 and/or developer terminal via the technology provider terminal. In this case, the tool is preferably in the form of an install file that may be installed on the developer terminal and/or development device or an executable file. The instructional information is in the form of a document, image, video, audio, or other file that may be visually/auditorily identified by the developer via the developer terminal and/or development device.

ii) Providing Offline

The technology provider may directly provide, offline, the development device used for collaborative development to the developer. Since the development device includes the technology provider's know-how, it needs to be prevented from loss or damage. Thus, the technology provider may provide it to the developer using personnel and/or device for transportation and installation.

Further, the development tool kit that may be operated on the developer terminal and/or development device may be stored in a portable storage device and provided to the developer for the above reasons. The portable storage device may be selected from among various types including external hard drives, universal serial bus (USB) drives, or compact discs (CDs).

iii) Providing with it Installed on Development Device

Among the technology provider's development tool kits, some development tool kits used for the development device may be pre-installed on the collaborative development and be provided to the developer.

3) Entity to Use Development Tool Kit

The development tool kit may be downloaded from the collaborative development system 1000 or technology provider terminal (stand-alone type) or be used in an online service type (cloud type) by accessing the tool kit service module 135 in the collaborative development server 100.

The development tool kit provided from the collaborative development system 1000 is allowed to be used by the developer terminal or development device according to the following implementation examples.

i) Use of Development Tool Kit by Developer Terminal

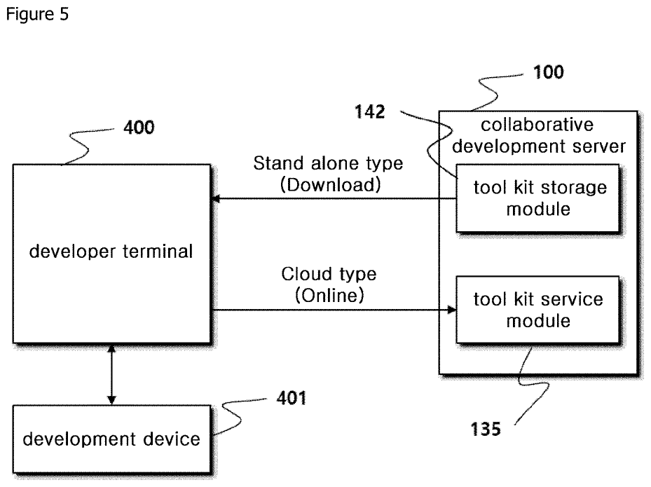

The entity to use the development tool kit may be the developer terminal. FIG. 5 illustrates an example scheme in which the developer terminal accesses the development tool kit stored in the collaborative development server 100.

FIG. 5 is a view illustrating a configuration of accessing a development tool kit stored in a collaborative development server by a developer terminal or development device according to the disclosure. As shown in FIG. 5, the developer terminal 400 accesses the tool kit storage module 142 of the collaborative development server 100 and downloads the development tool kit (stand-alone type).

Further, the developer terminal 400 accesses the tool kit service module 135 of the collaborative development server 100 and uses the development tool kit in an online service type (cloud type).

ii) Use of Development Device's Development Tool Kit Via Developer Terminal

The entity to use the development tool kit may be the development device. However, the development device does not directly access the collaborative development server 100 but accesses the collaborative development server 100 indirectly via the developer terminal linked to the collaborative development server 100 to use the development tool kit.

The scheme of accessing the development tool kit stored in the collaborative development server 100 by the development device may be described as follows. According to another embodiment, the development device may indirectly access the tool kit storage module 142 of the collaborative development server 100 via the developer terminal and download and use the development tool kit.

Further, the development device may access the tool kit service module 135 of the collaborative development server 100 via the developer terminal and use the development tool kit in an online service type (cloud type).

In other words, where the development device is capable of communication connection with the developer terminal but is incapable of connecting to an external communication network in another embodiment, the development device may indirectly access the collaborative development server 100 via the developer terminal to download the development tool kit or receive it in an online service type.

iii) Use of Development Tool Kit by Development Device

The entity to use the development tool kit may be the development device. Another scheme of accessing the development tool kit stored in the collaborative development server 100 by the development device, according to another embodiment, may be described below.

According to another embodiment, the development device may access the tool kit storage module 142 of the collaborative development server 100 to download and use the development tool kit.

Further, the development device may access the tool kit service module 135 of the collaborative development server 100 and use the development tool kit in an online service type (cloud type).

In other words, according to another embodiment, the development device may directly access the collaborative development server 100 via a communication network to download the development tool kit or be served the development tool kit in an online service type.

It is preferable to previously register the development device provided to the developer as a device used for collaborative development, in the development device management module 134 of the collaborative development server 100. By doing so, the condition of the development device used by the developer and/or the developer terminal may be monitored. Further, the log generated by the development device may be received and stored by the collaborative development system 1000. The log is stored in the collaborative storage module 141 of the database unit 140 by the data management module 131.

The disease region storage module 143 stores various pieces of information about the diseases that are currently breaking out, and/or have broken out, all over the world. The stored information includes the name (definition) of each disease, disease classification code, disease pathogens, disease transmission routes, disease outbreak regions (current and past), various descriptions of disease (e.g., incubation period, symptoms, treatment and care, prevention, and diagnosis methods), or disease keywords. The disease information stored in the disease region storage module 143 also includes contagious diseases and hereditary diseases. Each disease stored in the disease region storage module 143 may include the following information and, as necessary, relevant additional information also.

i) Name of Disease

Diseases may be classified into contagious diseases and hereditary diseases, and the names of the contagious diseases and hereditary diseases classified are stored.

For example, the contagious diseases include cholera, typhoid fever, paratyphoid fever, shigellosis, hepatitis A, diphtheria, measles, Streptococcus pneumoniae, malaria, anthrax, AIDS (acquired immune deficiency syndrome), influenzas, plague, dengue fever, Ebola virus, MERS (Middle East Respiratory Syndrome Coronavirus), avian Influenza, yellow fever, polio, etc.

The hereditary diseases include sickle-cell disorder, albinism, Wilson's disease, Huntington's disease, Fabry's disease, melas syndrome, cat's cry syndrome, WARG syndrome, Wolf-Hirschhorn syndrome, Patau syndrome, down syndrome, turner syndrome, Klinefelter syndrome, epilepsy, nearsightedness, diabetes mellitus, or other various diseases.

ii) Disease Region

Disease outbreaks may be classified region-wise. The regions may be classified into, e.g., geographical regions, climatological regions, and/or administrative regions.

For example, the diseases may be classified based on administrative regions, into, e.g., diseases that break out primarily in the African Continent, specifically in South America, mainly in Asia, specifically in India, or specifically in Hunan, China. Or, the diseases may be classified based on climatological regions, into, e.g., diseases that break out specifically in the Mediterranean coastline, mainly in the subtropics, or specifically in tropical climate. The diseases may also be classified, based on the geographical regions, into, e.g., diseases that break out mainly in the regions over latitude 24 degrees N and in the regions between latitude 15 degrees N and 15 degrees S.

In particular, each country's centers for disease control and prevention (CDC) that controls quarantine for travelers provides information about the regions where contagious diseases are breaking out.

According to an embodiment, Ebola virus is spreading in the Democratic Republic of the Congo.

According to another embodiment, the MERS is breaking out in Saudi Arabia, the United Arab Emirates, Oman, and Kuwait. As necessary, Yemen, Qatar, Bahrain, Jordan, Iran, and Lebanon, which are neighbor countries of the polluted regions may be additionally designated.

According to another embodiment, avian Influenza (H5N1, H7N9) is spreading in five provinces of China, including Guangdong, Guangxi, Yunnan, Jiangsu, and Hunan.

According to another embodiment, yellow fever is spreading in Cameroon, Democratic Republic of the Congo, Congo, Angola, Benin, Burkina Faso, Burundi, Central African Republic, Chad, Ivory Coast, Equatorial Guinea, Ethiopia, Gabon, Gambia, Ghana, Guinea, Guinea-Bissau, Kenya, Liberia, Mali, Mauritania, Niger, Nigeria, Siena Leone, Senegal, South Sudan, Sudan, Togo, and Uganda on the African Continent, Argentina, Bolivia, Brazil, Colombia, Ecuador, French Guiana, Guyana, Panama, Paraguay, Peru, Suriname, Trinidad and Tobago, and Venezuela in the Americas.

In another embodiment, cholera is spreading in Yemen, Philippines, and India in Asia, Cameroon, Congo, Angola, Kenya, Malawi, Niger, Nigeria, Uganda, Tanzania, Somalia, Mozambique, Zambia, Zimbabwe, and Algeria on the African Continent.

In another embodiment, polio is spreading in Pakistan, and Afghanistan in Asia, Democratic Republic of Congo, Kenya, Niger, Nigeria, Tanzania, and Papua New Guinea on the African Continent.

In another embodiment, plague has new cases in Madagascar on the African Continent.

In the above-described embodiments, although the outbreak regions of each disease are stored with the continent and country names divided based on the administrative regions, they may be classified and stored based on the geographical regions and/or climatological regions, as necessary.

According to an embodiment, in the geographical regions, the disease regions may be displayed with latitudes and longitudes or GPS coordinates, or the entire globe may be divided into lattices with a predetermined area of size and displayed with lattice addresses or may otherwise be displayed by other geographical display methods.

According to another embodiment, the climatological regions may be displayed as climate regions resultant from dividing disease regions by various criteria. For example, the disease regions may be divided into tropical climate, dry climate, temperate climate, microthermal climate, and polar climate regions depending on the temperature of the region.

Each of the climates divided into depending on the temperature is sub-divided. For example, the tropical climate is sub-divided into tropical rainforest climate (Af), tropical monsoon climate (Am), wet and dry climate (Aw), and tropical savanna climate (As). The dry climate is sub-divided into desert climate (BS) and steppe climate (BW). The temperate climate is sub-divided into humid subtropical climate (Cfa), west coast oceanic climate (Cfb, Cfc), temperate with dry winter climate (Cw), and Mediterranean climate (Cs). The microthermal climate is sub-divided into humid continental climate (DO, continental dry winter climate (Dw), and highland Mediterranean climate (Ds). Lastly, the polar climate is sub-divided into tundra climate (ET) and ice cap climate (EF).

In another embodiment, in the administrative regions, the disease regions may be displayed as administrative regions. For example, the disease regions may be classified into continents, countries, cities, or such regions.

The information about the disease outbreak regions stored in the disease region storage module 143 may be obtained via the information provided from the World Health Organization (WHO) and/or each country's centers of disease control and prevention (CDC).

For example, the WHO or CDC provides information about the diseases that globally break out. The provided information includes the definition and classification of each disease, the nationwide and worldwide outbreaks, causes, transmission routes, symptoms, diagnosis and treatment, patient care, prevention, and infected region information. The disease region storage module 143 stores the disease outbreak regions via the disease information and may also store disease keywords via the relevant disease information.

iii) Outbreak Time of Disease

A disease ceases to spread for a predetermined period but, after that, breaks out again. Further as an infected person leaves the outbreak reason, a new case may be found in another region. In such a case, even with a long distance between the two regions and different outbreak times, the regions may be shown as the outbreak of the same disease.

In contrast, although another region has a new case as the infected person moves from the outbreak region, the disease may mutate. In this case, they may be determined to be the same disease, but a need may exist for developing separate reagents for determining whether they are infected.

The disease region storage module 143 stores diseases per disease name, disease outbreak region, and disease outbreak time and also stores additional information, e.g., disease keywords for identifying the diseases. The disease keyword may be compared with the name of organism containing the target nucleic acid, the name of gene containing the target nucleic acid, the sequence of the target nucleic acid, the intended use of the reagent for detection of the target nucleic acid, name of disease, or classification symbol of disease included in the feature information for the target nucleic acid, thereby allowing for determination as to what disease the feature information for the target nucleic acid is specified as.

Thus, the disease keyword is information for identifying the disease and specifies the disease by comparison with the feature keyword extracted from the target nucleic acid feature information in the request for collaborative development information, so that one or more regions where the disease breaks out may be selected as disease regions.

Each disease keyword is composed of various pieces of information for specifying the disease.

In an embodiment, the disease keywords for MERS may include Middle East Respiratory Syndrome Coronavirus (MERS), Middle East Respiratory Syndrome, Coronavirus, Acute Respiratory Disease, MERS-CoV, Beta Coronavirus, HCoV-EMC/2012, Glade A, Glade B, Jordan-N3/2012, Vero Cells (Vero cell), human beta coronavirus, HCoV-OC43, HCoV-HKU1, new coronavirus, Saudi SARS, specific gene (UpE, ORF1a, ORF1b, N), affected area (Saudi Arabia, UAE, Oman, Kuwait, Yemen, Qatar, Bahrain, Jordan, Lebanon, Iran), major clinical symptoms (fever, coughing, difficulty in breathing, headache, chills, sore throat, runny nose, muscle pain, anorexia, nausea, vomiting, abdominal pain, diarrhea, pneumonia), complications (respiratory failure, Pulmonary shock, multiple organ failure), test findings (leukopenia, lymphocytopenia, thrombocytopenia, LDH elevation).

In another embodiment, the disease keywords for Ebola virus may include Ebola virus, viral hemorrhagic fever, Ebola-Zaire, Ebola-Sudan, Ebola-Ivory Coast, Ebola-Reston, Ebola hemorrhagic fever (EHF), phyllo virus family, zoophilic virus, diseased area (Central Africa, Democratic Republic of the Congo, Sudan), genetic sequence (3′-leader-NP-VP35-VP40-GP/sGP-VP30-VP24-L-tail-5), major clinical symptoms (headache, fever, myalgia, nausea, vomiting), Diarrhea, cough, chest pain, skin rash, edema, ocular hyperemia, sore throat), test findings (reduction of white blood cells, increased platelet count, increased liver enzymes).

In another embodiment, the disease keywords for cholera may include cholera (Vibrio cholerae O1 or V. cholerae O139), V. cholerae Bengal, ICD-10 A00, Vibrionaceae, Cholerae toxin, V. cholerae classical type, V. cholera E1 tor type, Inaba subtype, Ogawa subtype, Hikojima subtype, fish and shellfish infection, oral infection, major clinical (diarrhea, vomiting, dehydration, hypovolemic shock), complications (renal failure, dehydration, electrolyte imbalance).

In another embodiment, the disease keywords for plague may include plague, Yersinia pestis, gram-negative bacteria, sun fest (bubo plague), septicemic plague, pneumonic plague, pharyngeal fest, meninges fest, insufficiency fest, black plague, plague, beta receptor blockade, major clinical (chills, fever, muscle pain, joint pain, headache, hemorrhagic purulent inflammation, lymph node hemorrhagic inflammation, nausea, vomiting, diarrhea, hemorrhagic spots, decreased kidney function, shock, acute respiratory failure syndrome, general weakness, Shortness of breath, cough, sputum, chest pain, hemoptysis, respiratory failure, cardiovascular failure, ischemic necrosis), complications (disseminated intravascular coagulation, acute respiratory failure, meningitis).

According to an embodiment, the database unit 140 may not include the disease region storage module 143.

II. Computer-Implemented Method for Collaborative Development of Reagent for Detecting Target Nucleic Acid

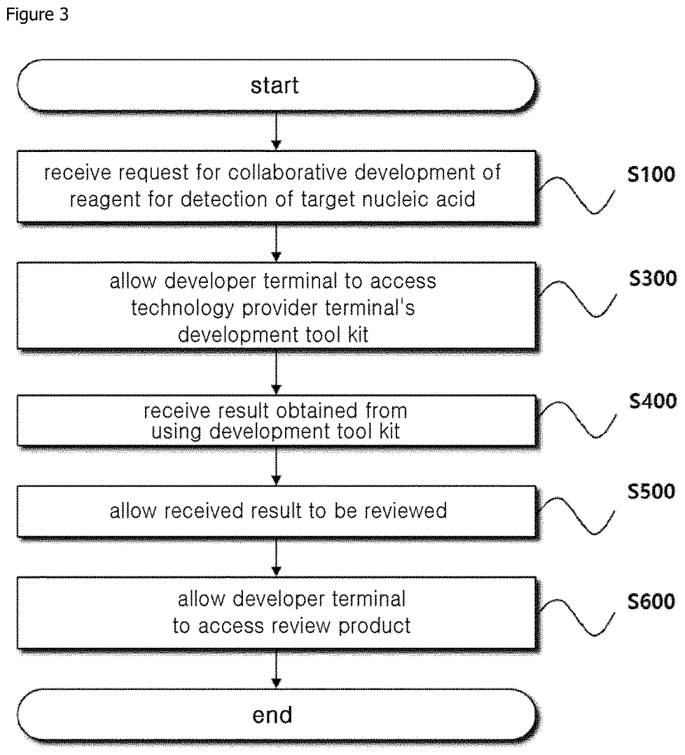

One aspect of a collaborative development method according to the disclosure is schematically shown in FIG. 3. The collaborative development method according to the disclosure is stepwise described below in detail.

Step (a): Receiving Request for Collaborative Development

First, a request for collaborative development of a reagent containing an oligonucleotide for detection of a target nucleic acid is received from a requester terminal (S100). The request for collaborative development includes feature information about to the target nucleic acid to be detected using the reagent. The collaborative development system receives the request for collaborative development and assigns a receipt number (S110).

The disclosure relates to collaborative development of a reagent for detection of a target nucleic acid, which is performed by a technology provider(s) and a developer(s).

As used herein, the term “collaborative development” means a joint contribution by the technology provider and the developer in the course of developing a reagent for detection of a target nucleic acid. In particular, the term “collaborative development” herein encompasses providing a development technique by the technology provider and testing by the developer in the course of developing a reagent for detection of a target nucleic acid.

In the collaborative development, the joint contribution by the technology provider and the developer does not necessarily mean that the technology provider and the developer equally contribute to the development. In other words, the collaborative development according to the disclosure should be interpreted as encompassing different degrees of contributions by the technology provider and the developer.

The degrees of contributions by the technology provider and the developer attending the collaborative development may be varied depending on a number of factors, such as the technology provider's and the developer's technical skills, resources, development traits, or contracts. For example, where the technique provided by the technology provider is limited, the degree of contribution by the technology provider may be lower than that by the developer. In contrast, where the technique provided by the technology provider is comprehensive, the degree of contribution by technology provider may be higher than that by the developer. As another example, the degrees of contributions by the technology provider and the developer may be adjusted by an agreement or contract between the technology provider and the developer.

Although there may be the participation of other third parties, e.g., the requester, evaluator, manufacturer, or investor in the collaborative development according to the disclosure, their participation is not essential to the collaborative development according to the disclosure as the third parties do not directly contribute to the development of the reagent.

The reagent to be developed according to the disclosure means a mixture of components used to detect the target nucleic acid. The reagent contains components that play a role in amplifying/detecting the target nucleic acid, such as an oligonucleotide (e.g., a primer and/or probe) that is hybridized specifically to the target nucleic acid, enzymes (e.g., nucleic acid polymerase, nucleic acid restriction enzyme, polymerase with restriction enzyme activity), a label, and other necessary components (a buffer, metal ions, dNTPs, salts, etc.). Examples of the components of the reagent are disclosed in WO 2015/147412, the disclosure of which is incorporated by reference herein in its entirety.

According to an embodiment, the reagent to be developed contains an oligonucleotide for detecting the target nucleic acid. In the disclosure, the oligonucleotide may be a primer and/or a probe.

According to an embodiment, the reagent to be developed contains a label for detecting the target nucleic acid.

According to an embodiment, the reagent to be developed contains a labeled primer and/or labeled probe for detecting the target nucleic acid.

According to an embodiment, the reagent to be developed contains an enzyme.

According to an embodiment, the reagent to be developed contains a buffer.

In particular, the reagent to be developed is for multiplex detection (detection of a plurality of target nucleic acids).

The term “primer” as used herein refers to an oligonucleotide, whether occurring naturally or produced synthetically, which is capable of acting as a point of initiation of synthesis when placed under conditions in which synthesis of primer extension product which is complementary to a nucleic acid strand (template) is induced, i.e., in the presence of nucleotides and an agent for polymerization such as DNA polymerase and at a suitable temperature and pH. The primer is preferably single stranded for maximum efficiency in amplification. Preferably, the primer is an oligodeoxyribonucleotide. The primer of this invention can be comprised of naturally occurring dNMPs (i.e., dAMP, dGM, dCMP and dTMP), modified nucleotide or non-natural nucleotide. The primer can also include ribonucleotides. The primer must be sufficiently long to prime the synthesis of extension products in the presence of the agent for polymerization. The exact length of the primers will depend on many factors, including temperature, application and source of primer.

“Probe” is a single-chain nucleic acid molecule including a sequence substantially complementary to a target nucleic acid sequence. The term “complementary” is used herein to mean that primers or probes are sufficiently complementary to hybridize specifically to a target nucleic acid sequence under the designated annealing conditions or stringent conditions, encompassing the terms “substantially complementary” and “perfectly complementary”, preferably perfectly complementary. Preferably, the probe is a single-chain deoxyribonucleotide. The primer used in the disclosure may include a naturally occurring dNMPs (i.e., dAMP, dGMP, dCMP and dTMP), modified nucleotide, or non-natural nucleotide. The probe can also include ribonucleotides. The probe may include a label capable of generating a signal for detecting the target.

As used herein, the phrase “(single or multiplex) target nucleic acid detection” means that one target nucleic acid is detected in one reaction. The target nucleic acid detection includes not only amplifying the target nucleic acid and detecting the amplification product (amplicon) but also detecting hybridization events with oligonucleotides without amplifying the target nucleic acid.

As used herein, the phrase “multiplex target nucleic acid detection” means simultaneously detecting two or more target nucleic acids in one reaction. The multiplex target nucleic acid detection includes not only simultaneously amplifying two or more target nucleic acids and detecting the amplification product for each target but also detecting hybridization events with oligonucleotides without amplifying the two or more target nucleic acids.

The amplification of a target nucleic acid molecule may be performed by various primer-involved nucleic acid amplification methods known in the art. Specifically, the amplification of a target nucleic acid is performed according to polymerase chain reaction (PCR), which is disclosed in U.S. Pat. Nos. 4,683,195, 4,683,202, and 4,800,159. Other examples are ligase chain reaction (LCR, U.S. Pat. Nos. 4,683,195 and 4,683,202; and PCR Protocols: A Guide to Methods and Applications (Innis et al., eds, 1990)), strand displacement amplification (SDA, Walker, et al. Nucleic Acids Res. 20(7):1691-6 (1992); and Walker PCR Methods Appl. 3(1):1-6 (1993)), transcription-mediated amplification (Phyffer, et al., J. Clin. Microbiol. 34:834-841 (1996); and Vuorinen, et al., J. Clin. Microbiol. 33:1856-1859 (1995)), nucleic acid sequence-based amplification (NASBA, Compton, Nature 350(6313):91-2 (1991)), rolling circle amplification (RCA, Lisby, Mol. Biotechnol. 12(1):75-99 (1999); and Hatch et al., Genet. Anal. 15(2):35-40 (1999)), and Q-Beta Replicase (Lizardi et al., BiolTechnology 6:1197 (1988)). The target nucleic acid detection according to the disclosure may be performed by real-time PCR (see U.S. Pat. Nos. 5,210,015, 5,538,848 and 6,326,145).

As used herein, the phrase “collaborative development of a reagent for detection of a target nucleic acid” includes establishing a composition of the reagent for detection of the target nucleic acid by the collaboration of the technology provider and the developer.

According to an embodiment, the collaborative development of the reagent for detection of the target nucleic acid includes designing candidate oligonucleotides to be included in the reagent for detection of the target nucleic acid.

According to an embodiment, the collaborative development of the reagent for detection of the target nucleic acid includes testing the performance of oligonucleotides (in particular, candidate oligonucleotides).

According to an embodiment, the collaborative development of the reagent for detection of the target nucleic acid includes selecting the optimal oligonucleotide or the optimal combination of oligonucleotides from among the candidate oligonucleotides.

In this step, a request for collaborative development is received from the requester terminal.

Requester/Requester Terminal

As used herein, the term “requester” means an entity that proposes the necessity of detecting a specific organism (in particular, a pathogen) or a target nucleic acid therefor via the collaborative development system of the disclosure and may be interchangeably used with the term “proposer.”

As used herein, the term “requester terminal” means a terminal operated by the requester.

In the disclosure, the requester terminal is used by the requester to interact with the collaborative development system according to the disclosure. The requester terminal may be a computer, such as a personal computer (PC), desktop computer, laptop computer, notebook, smartphone, or tablet PC, or any device including the same. Computer is a device including one or more general-purpose or special-purpose processor, memory, storage, and networking component (wired or wireless). The device executes an operating system (OS), e.g., Microsoft Windows-compatible OS, Apple OS X or iOS, Linux, or Google Android OS. According to an embodiment, the requester terminal may use a web browser, such as Microsoft Internet Explorer, Mozilla Firefox, Google Chrome, Apple Safari, and/or Opera, as an interface for interaction with the collaborative development system of the disclosure. According to an embodiment, the requester terminal may execute a dedicated application for accessing the collaborative development system.

The above description of the terminal is also applied to the developer terminal and technology provider terminal disclosed below.

According to an embodiment, the requester is an individual.

According to another embodiment, the requester is an organization, such as a university, research center, enterprise, or hospital.

According to an embodiment, the requester is a single one. For example, a single requester may provide a request for sufficient development of a reagent for detection of the target nucleic acid, e.g., sufficient feature information about a target nucleic acid.

According to another embodiment, the requester is multiple ones. While a request from a single requester may be insufficient to develop a reagent for detection of the target nucleic acid, a combination of a request from one requester with a request from other requester may be sufficient to develop a reagent for detection of the target nucleic acid. For example, a request from a first requester may include feature information about a first target nucleic acid, and a request from a second requester may include feature information about a second target nucleic acid. A combination of the request from the first requester and the request from the second requester may achieve sufficient development of a reagent for detection of two target nucleic acids. However, where the first requester and the second requester provide feature information about the same target nucleic acid, the two requesters may be regarded as the same requester.

In the disclosure, information about the requester may, or may not, be registered in the collaborative development system of the disclosure.

The identity between the requester in this step and the developer and technology provider in other steps may be explained as follows:

According to an embodiment, the requester is identical to the developer. For example, after submitting a request for collaborative development, the requester may also participate in the collaborative development as a developer.

In another embodiment, the requester differs from the developer. For example, after submitting a request for collaborative development, the requester may not participate in the collaborative development.

According to an embodiment, the requester is identical to the technology provider.

For example, after submitting a request for collaborative development, the requester may also participate in the collaborative development as a technology provider.

In another embodiment, the requester differs from the technology provider. For example, after submitting collaborative development, the requester may not participate in the collaborative development.

The above-described request for collaborative development may be received in various manners.

According to an embodiment, the request for collaborative development is received in a manner such that the requester terminal accesses the webpage and enters the content of the request for collaborative development.

According to another embodiment, the request for collaborative development is received in a manner such that a file for creating the content of the request for collaborative development is provided to the requester terminal via a request management module, and the content of the request for collaborative development entered to the requester terminal by the requester is received in the form of a file.

The method according to the disclosure may further include assigning a unique identifier to the request for collaborative development received from the requester terminal.

The collaborative development system generates unique identifiers for the developer and/or technology provider participating in collaborative development and provides the identifiers to each developer terminal and/or technology provider terminal. The identifiers may be used for identifying the developer and technology provider or the developer terminal and technology provider terminal, use authentication for use in, and access to, the development device or development tool kit used for collaborative development, and encryption of the results (including documents) generated by the developer and technology provider.

Thus, the identifiers include, e.g., IDs and passwords of participants being matched to the project number in order to identify the developer and the technology provider. The identifiers also include, e.g., a certificate for access to, and approval of use of, the development device or the development tool kit. The identifiers include an encryption key or public key for encrypting the generated data. The identifier includes a decryption (or decoding) key or private key for the developer or technology provider attending the collaborative development to view the encrypted result.

The identifier may be given to various objects, such as the preliminarily evaluated request for collaborative development, substantially evaluated request for collaborative development, the development tool kit provided from the technology provider, the result obtained from using the tool kit, the review product by the technology provider, and verification result, as well as the received request for collaborative development.

In the disclosure, the request for collaborative development received from the requester includes feature information about a target nucleic acid to be detected using the reagent.

According to an embodiment, the feature information about the target nucleic acid includes: (i) a name of an organism containing the target nucleic acid; (ii) a name of a gene containing the target nucleic acid; (iii) a sequence of the target nucleic acid; (iv) an intended use of the reagent; (v) a name of a disease to be detected with the reagent; and/or (vi) a classification symbol of the disease to be detected with the reagent.

Hereinafter, the feature information about the target nucleic acid is described:

(i) Name of Organism Containing Target Nucleic Acid

The name of the organism may be included in the feature information about the target nucleic acid. Where the requester is aware of the name of the organism to be detected, the requester may include the name of the organism in the request for collaborative development.

The organism name may include one genus, species, subspecies, subtype, genotype, serotype, strain, isolate, and cultivar.

The organism name includes the name according to: (1) the International Code of Nomenclature for algae, fungi, and plants; (2) the International Code of Zoological Nomenclature; (3) the International Code of Nomenclature of Prokaryotes; (4) the International Code of Nomenclature for Cultivated Plants; (5) the International Code of Nomenclature of Bacteria and Viruses; or (6) the International Code of Phytosociological Nomenclature. Generally, the organism name includes the name according to binominal nomenclature including genus and species. The organism name includes other various names, such as general name, alias or acronym.

Besides the organism name, other information regarding the organism, e.g., the organism's taxonomy ID, source, and relevant documents may further be included in the feature information about the target nucleic acid.

(ii) Name of Gene Containing Target Nucleic Acid

The name of the gene may be included in the feature information about the target nucleic acid. Where the requester is aware of the gene inherent to the organism to be detected, the requester may include the name of the gene in the request for collaborative development.

Besides the gene name, other information about the gene, e.g., the gene's source, accession number, and relevant documents may further be included in the feature information about the target nucleic acid.

(iii) Sequence of Target Nucleic Acid

The sequence of the target nucleic acid may be included in the feature information about the target nucleic acid. Where the requester is aware of the sequence of the target nucleic acid contained in the organism to be detected, the requester may include the target nucleic acid sequence in the request for collaborative development.

Besides the target nucleic acid sequence, other information about the target nucleic acid sequence, e.g., the target nucleic acid's source, accession number, and relevant documents may be included in the feature information about the target nucleic acid.

(iv) Intended Use of Reagent

The intended use of the reagent for detection of the target nucleic acid may be included in the feature information about the target nucleic acid.

The intended use of the reagent may include identification of diseases, disorders, conditions, symptoms, or syndromes, associated with the target nucleic acid to be detected using the reagent, i.e., known to be caused or to be highly likely to be caused by the organism containing the target nucleic acid.

(v) Name of Disease to be Caused by Organism Containing Target Nucleic Acid

The name of the disease to be caused by the organism containing the target nucleic acid may be included in the feature information about the target nucleic acid.

Examples of the disease may include, but are not limited to, cholera, typhoid fever, paratyphoid fever, shigellosis, hepatitis A, diphtheria, measles, Streptococcus pneumoniae, malaria, anthrax, AIDS (acquired immune deficiency syndrome), influenza, plague, dengue fever, Ebola virus, MERS (Middle East Respiratory Syndrome Coronavirus), avian influenza, yellow fever, polio or other infectious diseases; and sickle-cell disorder, albinism, Wilson's disease, Huntington's disease, Fabry's disease, melas syndrome, cat's cry syndrome, WAGR syndrome, Wolf-Hirschhorn syndrome, Patau syndrome, down syndrome, turner syndrome, Klinefelter syndrome, epilepsy, nearsightedness, diabetes mellitus, or other hereditary diseases.

(vi) Classification Symbol of Disease to be Caused by Organism Containing Target Nucleic Acid

The classification symbol of the disease detected by the reagent for detection of the target nucleic acid may be included in the feature information about the target nucleic acid. The classification symbols of disease may be determined based on the ICD (International Classification of Diseases and Related Problems) of the WHO or each country's ICD. For example, the Republic of Korea adopts the Korean standard classification of diseases (KCD). Examples of ICD-10 and the KCD are found in the rest of the disclosure.

In the disclosure, the request for collaborative development received from the requester may include additional information that may be referenced in developing the reagent for detection of the target nucleic acid, as well as the feature information about the target nucleic acid set forth in (i) to (iv) above. Examples of the additional information may include the sample applied, extraction method, detecting device, method of interpretation of test results, configuration of product, turnaround time (TAT), performance requirements, development period, and development costs.

In an embodiment, the method according to the disclosure further includes transmitting the request for collaborative development received from the requester terminal to the technology provider terminal. The transmission of the request for collaborative development to the technology provider terminal is intended for creating an environment in which the technology provider may recognize the collaborative development and response to the request.

The method of the disclosure may further include evaluating the request for collaborative development between step (a) and step (b). The evaluation step is described below.

Evaluation Step

The step of evaluating the request for collaborative development, as included in the method according to the disclosure, may be performed in various manners.

In light of the entity to perform the evaluation, the step of evaluating the request for collaborative development included in the method according to the disclosure may be performed by an evaluator or by an evaluation management module of the collaborative development system.

According to an embodiment, the method according to the disclosure further includes transmitting a request for evaluation to an evaluator terminal in response to the request for collaborative development and receiving an evaluation result of the feasibility of collaborative development from the evaluator terminal.

The evaluator may be an individual or an organization, e.g., school, company, or hospital. The evaluator may be a single evaluator or multiple evaluators including e.g., a first evaluator, a second evaluator, and a third evaluator. The evaluator may be a chosen one. As an example, the evaluator is a single evaluator chosen from among a plurality of evaluators. As another example, the evaluator may be multiple evaluators chosen from among a plurality of evaluators.

The selection of the evaluator may be performed by the developer or technology provider, or according to an agreement between the developer and the technology provider, or by the evaluation management module of the collaborative development system.

The evaluator terminal may include the technology provider terminal. As an example, where the evaluator is a single one, the evaluator terminal may be a first technology provider terminal. As another example, where the evaluation is multiple ones, the evaluator terminal may consist of a first evaluator terminal, a second evaluator terminal, and a first technology provider terminal.

According to another embodiment, the method according to the disclosure further includes evaluating the feasibility of collaborative development by an evaluation management module of the collaborative development system in response to the request for collaborative development.

The evaluation management module of the collaborative development system may include criteria for evaluating the feasibility of collaborative development.

As used herein, the term “criteria” denotes evaluation elements used for evaluation, such as evaluation items, scores, and weights. Evaluation items included in the criteria may largely include technological feasibility, business feasibility, marketability, originality, cost, and difficulty in development. Score included in the criteria is a range of points selectable per evaluation item by the evaluator. Weight included in the criteria is a value for adjusting the evaluation score in proportion to the importance of each evaluation item. The criteria may be ones previously determined or may be criteria according to the results trained based on artificial intelligence (AI) using deep learning or machine learning. The evaluation management module may score the collaborative development feasibility according to the criteria.

In light of evaluation schemes, the step of evaluating the request for collaborative development included in the method according to the disclosure may include preliminary evaluation and substantial evaluation. The preliminary evaluation includes formal evaluation of the request for collaborative development or brief substantial evaluation, e.g., evaluation as to whether the request for collaborative development contains necessary content. The substantial evaluation includes evaluation on the merits of the collaborative development, e.g., evaluation of the feasibility of collaborative development.

The preliminary evaluation and the substantial evaluation may be performed by the evaluator and the evaluation management module of the collaborative development system. As an example, the preliminary evaluation and the substantial evaluation both may be performed by the evaluator. As another example, the preliminary evaluation and the substantial evaluation both may be performed by the evaluation management module. As another example, after the preliminary evaluation is performed by the evaluator, the substantial evaluation may be performed by the evaluation management module of the collaborative development system. As another example, after the preliminary evaluation is performed by the evaluation management module of the collaborative development system, the substantial evaluation may be performed by the evaluator. As another example, the preliminary evaluation and/or the substantial evaluation may be performed by the evaluator and the evaluation management module of the collaborative development system

The result of the preliminary evaluation may be, e.g., approval or supplementation. Approval here means that the request has passed the formal evaluation while supplementation denotes that the request needs to be formally supplemented.

The result of the substantial evaluation may be, e.g., approval or rejection. Approval here means that the requested collaborative development is proper to continue while rejection means that the requested collaborative development is improper to proceed. Where the collaborative development is approved, the method according to the disclosure may perform step (b) and, if the collaborative development is rejected, the method according to the disclosure may include ceasing to proceed by the collaborative development system.

Step (b): Access Development Tool Kit by Developer Terminal

Subsequently, the developer terminal is allowed to access the technology provider's development tool kit for collaborative development (S300). The development tool kit includes a performance test tool for the oligonucleotide included in the reagent for detection of the target nucleic acid. The performance test tool includes one or more of software, a device, and instructional information.

Developer/Developer Terminal

As used herein, the term “developer” refers to an entity that contributes to collaborative development of a reagent for detection of the target nucleic acid by a method according to the disclosure, specifically an entity in charge of most of a test for development. As used herein, the term “developer terminal” means a terminal operated by the developer.

As used herein, the developer may be, without limitation, a person who has no experience of development of a reagent for detection of the target nucleic acid, has no experience of development of a reagent for detection of the target nucleic acid using the technique provided by the technology provider, has an test environment, is easily accessible to a clinical sample, or otherwise needs collaborative development.

According to an embodiment, the developer terminal is a terminal of a developer who has already obtained or is able to obtain a clinical sample containing, or suspected of containing the target nucleic acid.

The term used herein “sample” refers to any cell, tissue, or fluid from a biological source, or any other medium that can advantageously be evaluated according to this disclosure, including virus, bacteria, tissue, cell, blood, serum, plasma, lymph, milk, urine, feces, ocular fluid, saliva, semen, brain extracts, spinal cord fluid (SCF), appendix, spleen and tonsillar tissue extracts, amniotic fluid, ascitic fluid and non-biological samples (e.g., food and water). In addition, the sample includes natural-occurring nucleic acid molecules isolated from biological sources and synthetic nucleic acid molecules.

In particular, as used herein, the term “clinical sample” denotes a sample containing, or suspected of containing the target nucleic acid and which is used to measure and verify the performance of the reagent (in particular, an oligonucleotide) for detection of the target nucleic acid. The clinical sample originates from a target, preferably a human being, more preferably a human patient. Where the developer obtains or is able to obtain the clinical sample, the technology provider who is unable to obtain the clinical sample may provide his know-how to the developer, thereby allowing for development of the reagent for detection of the target nucleic acid using the clinical sample.

According to an embodiment, the developer terminal is a terminal of a developer located in the region where the clinical sample may be taken out or moved.

According to an embodiment, the developer terminal is a terminal of a developer located in the region where there is a facility capable of dealing with the clinical sample.

According to an embodiment, the developer terminal is a terminal of a developer who is located in a region where a disease associated with the target nucleic acid is breaking out or has broken out. The developer may be selected depending on whether he is located in a disease region.

According to an embodiment, the developer terminal is a terminal of a developer located in the region where a disease associated with the target nucleic acid is breaking out or has broken out and obtaining or able to obtain the sample containing or suspected of containing the target nucleic acid.

As used herein, the phrase “disease associated with target nucleic acid” means that the disease is caused by an organism (e.g., virus or bacteria) containing the target nucleic acid.

The selection of the developer/developer terminal is discussed more fully elsewhere in the specification.

According to an embodiment, the developer is an individual.

According to another embodiment, the developer is an organization, such as a university, research center, enterprise, or hospital.

According to an embodiment, the developer is a single one.

According to another embodiment, the developer is multiple ones. For example, a first developer and a second developer may share one collaborative development.

According to the disclosure, the developer may be a developer selected to perform collaborative development. In other words, the developer terminal is a terminal of a developer selected to perform the collaborative development from among a plurality of developers. As an example, the developer terminal is a terminal of a single developer selected to perform the collaborative development from among a plurality of developers. As another example, the developer terminal is a terminal of multiple developers selected to perform the collaborative development from among a plurality of developers.

The selection of the developer may be performed by the requester or technology provider, or according to an agreement between the requester and the technology provider, or by the participant selection module of the collaborative development system. The participant selection module provides a request for attending collaborative development to the selected developer and receives information indicating whether the developer is to attend the collaborative development from the developer terminal (S250). Upon receiving information indicating that the selected developer cannot attend collaborative development from the developer terminal, the participant selection module selects other developer and provides a request for attending the collaborative development.

The selection of the developer may be performed based on predetermined developer selection criteria. The developer selection criteria include, e.g., the developer's suitability for the request for collaborative development. Specifically, the developer selection criteria may include, e.g., the developer's test environment for collaborative development, test skills, test personnel, test experience, expertise, knowledge, and accessibility to the clinical sample.

As set forth above, the developer may be the requester or may differ from the requester.

In the disclosure, information about the developer may, or may not, be registered in the collaborative development system of the disclosure.

The above-described developer terminal accesses the technology provider's development tool kit for collaborative development.

The technology provider, as another entity for collaborative development, according to the disclosure, is described below.

Technology Provider/Technology Provider Terminal

As used herein, the term “technology provider” refers to an entity that contributes to collaborative development of a reagent for detection of the target nucleic acid by a method according to the disclosure, specifically a leading entity of the collaborative development. In the disclosure, the technology provider plays a role in providing an overall technology and test guide for collaborative development. As used herein, the term “technology provider terminal” means a terminal operated by the technology provider.

In the disclosure, the technology provider is one who owns the technique for developing the reagent for detection of the target nucleic acid. In the disclosure, the technology provider is one who has an experience of development of the reagent for detection of the target nucleic acid or has previously served as a technology provider in a similar collaborative development. In particular, in the disclosure, the technology provider is one with a tool kit for developing the reagent for detection of the target nucleic acid.

According to an embodiment, the technology provider is an individual. According to another embodiment, the technology provider is an organization, such as a university, research center, enterprise, or hospital.

According to an embodiment, the technology provider is a single one. According to another embodiment, the technology provider is multiple ones. For example, a plurality of technology providers may provide different tools included in the development tool kit for developing the reagent for detection of the target nucleic acid.

According to the disclosure, the technology provider may be one selected to perform the collaborative development. In other words, the technology provider terminal is the terminal of a technology provider selected to perform the collaborative development from a plurality of candidate technology providers. As an example, the technology provider terminal is the terminal of a single technology provider selected to perform the collaborative development from a plurality of candidate technology providers. As another example, the technology provider terminal is the terminal of multiple technology providers selected to perform the collaborative development from a plurality of candidate technology providers.

The selection of the technology provider may be performed by the requester or developer, or according to an agreement between the requester and the developer, or by the technology provider selection model included in the collaborative development system.

The selection of the technology provider may be performed by the requester or developer, or according to an agreement between the requester and the developer, or by the participant selection model included in the collaborative development system

The participant selection module provides a request for attending collaborative development to the selected technology provider and receives information indicating whether the technology provider is to attend the collaborative development from the technology provider terminal. Where the technology provider desires to attend the collaborative development, the technology provider's development tool kit is provided to the collaborative development system via the technology provider terminal.

Upon receiving information indicating that the selected technology provider cannot attend collaborative development from the technology provider terminal, the participant selection module selects other candidate technology provider and provides a request for attending the collaborative development.

The selection of the technology provider may be performed based on predetermined technology provider selection criteria. The technology provider selection criteria include, e.g., the technology provider's suitability for the request for collaborative development. Specifically, the technology provider selection criteria may include the technology provider's development experience, expertise, and knowledge for collaborative development.

In the disclosure, information about the technology provider may, or may not, be registered in the collaborative development system of the disclosure.

The above-described technology provider provides a development tool kit.

The development tool kit is described below.

Tool Kit for Developing Reagent for Detection of the Target Nucleic Acid

As used herein, the term “development tool kit” means a set of tools used to develop the reagent for detection of the target nucleic acid.

In the disclosure, the development tool kit includes a performance test tool for the oligonucleotide included in the reagent for detection of the target nucleic acid. The performance test tool includes one or more of software, a device, and instructional information.

Performance Test Tool for Oligonucleotide

The performance test tool for oligonucleotides denotes a tool that guides, instructs, helps, or facilitates the performance test for oligonucleotides.

According to an embodiment, the oligonucleotide performance test tool includes a tool for determining the sensitivity and/or specificity of oligonucleotides. More specifically, the oligonucleotide performance test tool includes a tool for determining the sensitivity of oligonucleotides, specificity of oligonucleotides, and/or interoligonucleotide interference (e.g., formation of a dimer between oligonucleotides).

In the disclosure, the sensitivity of oligonucleotide denotes the capability of the oligonucleotide to accurately determine a positive sample (e.g., a sample containing the target nucleic acid corresponding to the oligonucleotide) as positive. The sensitivity may be obtained by calculating the proportion of true positives in the positive sample. For example, the sensitivity may be calculated as follows:

Sensitivity (%)=(number of true positives)/(number of true positives+number of false negatives)

In the disclosure, the specificity of oligonucleotide denotes the capability of the oligonucleotide to accurately determine a negative sample (e.g., a sample not containing the target nucleic acid corresponding to the oligonucleotide) as negative. The specificity may be obtained by calculating the proportion of true negatives in the negative sample. For example, the specificity may be calculated as follows:

Specificity (%)=(number of true negatives)/(number of true negatives+number of false positives)

In the disclosure, the reagent for detection of the target nucleic acid to be collaboratively developed may typically include an oligonucleotide (e.g., a primer and/or probe) and may additionally include at least one of the following components: label, DNA polymerase, reverse transcriptase, dNTPs, Mg ions, KCl (or potassium acetate) and buffer. Among the above components, the other components than the oligonucleotide are non-specific to the target nucleic acid (i.e., irrelevant to the type of target nucleic acid) while the oligonucleotide is designed specifically to the target nucleic acid (e.g., to be hybridized with the target nucleic acid). Thus, the development of the reagent for detection of the target nucleic acid relies significantly on the performance of the oligonucleotide.

In this sense, the development of the reagent for detection of the target nucleic acid may largely include design of the candidate oligonucleotides, a performance test for the designed candidate oligonucleotides, and selection of an oligonucleotide based on the performance test.

In the disclosure, the performance test of the candidate oligonucleotides involves determining the ability of the candidate oligonucleotides, e.g., consisting of a forward primer, a reverse primer, and a probe, to detect the target nucleic acid. For example, in the case of the candidate oligonucleotides consisting of e.g., a first set of a first forward primer, a first reverse primer, and a first probe designed to detect a first target nucleic acid; a second set of a second forward primer, a second reverse primer, and a second probe designed to detect a second target nucleic acid; and a third set of a third forward primer, a third reverse primer, and a third probe designed to detect a third target nucleic acid, the performance test of the candidate oligonucleotides includes determining whether the first set detects only the first target nucleic acid, the second set detects only the second target nucleic acid, and whether the third set detects only the third target nucleic acid.

The performance test of the oligonucleotide for determination of the sensitivity or specificity of the oligonucleotide includes allowing the reagent containing the designed oligonucleotides to be reacted with a positive sample containing the target nucleic acid or a negative sample not containing the target nucleic acid to amplifying the target nucleic acid.

According to an embodiment, the device for determining the sensitivity or specificity of the oligonucleotide includes one or more devices selected from the group consisting of a nucleic acid extracting device, a nucleic acid dispensing device, and a nucleic acid detecting device. The devices may be selected by the developer and known in the art, selected by the technology provider and known in the art, or developed and provided by the technology provider. For example, the nucleic acid amplifying device and nucleic acid detecting device may be devices appropriate for implementing the technology provider's technique and may be well-known devices or devices developed by the technology provider. The device appropriate for implementing the technology provider's technique is a device that exhibits a performance suitable for the structure and design method of the oligonucleotide adopted in the technology provider's technique, and signal analysis methods.

According to an embodiment, the software for determining the sensitivity or specificity of the oligonucleotide includes instructions to drive the nucleic acid extracting device.

According to an embodiment, the software for determining the sensitivity or specificity of the oligonucleotide includes instructions to drive the dispensing device.

According to an embodiment, the software for determining the sensitivity or specificity of the oligonucleotide includes instructions to drive the nucleic acid amplifying device.

According to an embodiment, the software for determining the sensitivity or specificity of the oligonucleotide includes instructions to drive the nucleic acid detecting device.

According to an embodiment, the software for determining the sensitivity or specificity of the oligonucleotide includes an instruction for dispensing (localizing) the oligonucleotide in a designated position of a reaction vessel (e.g., reaction plate such as 96-well plate). More specifically, the software positions each reagent for detection of the target nucleic acid (including the oligonucleotide and the template) in a designated well of the reaction plate. The position of the reagent in a reaction plate may be determined by the technology provider or by an agreement between the technology provider and the developer. For example, the software may position a first reagent in the wells in row A-column 1 of 96-well plate (consisting of rows A to H and columns 1 to 12) and a second reagent in the wells in row A-column 2 of 96-well plate. Dispensing the reagents in designated positions is useful for analyzing the results of performance test on various reagents in a simplified and precise manner.

According to an embodiment, the instructional information for determining the sensitivity or specificity of the oligonucleotide includes processes and conditions of the performance test for the oligonucleotide. As used herein, the term “instructional information” is interchangeably used with “protocol,” “manual,” or “tutorial” for tests. In particular, the instructional information may include information about a sequential process of test, the amount of components used, the reaction condition, and the like. The instructional information may be in the form of a document, image, video, audio, or other file that may be visually/auditorily identified by the developer via the developer terminal and/or development device but not limited thereto.

According to an embodiment, the performance test tool for the oligonucleotide further includes a tool for selecting an oligonucleotide. The tool for selecting an oligonucleotide may include software for automatically selecting the optimal oligonucleotide from the oligonucleotide performance results, a device further used for the selection, or processes and conditions of tests for the selection.

The development tool kit according to the disclosure may further include a performance test tool for optimizing the reagent for detection of the target nucleic acid.

The performance test tool for the oligonucleotide is used for an oligonucleotide performance test to select the optimal oligonucleotide for the target nucleic acid while the performance test tool for optimizing the reagent for detection of the target nucleic acid is used to examine the effects of the other components than the oligonucleotide on the performance of the reagent and to optimize the other components. The optimization of the reagent for detection of the target nucleic acid includes determining appropriate types and amounts of the used components.

Similar to the oligonucleotide performance test tool, the performance test tool for optimizing the reagent for detection of the target nucleic acid may include software, a device, or instructional information.

The development tool kit according to the disclosure may further include one or more of the following: (i) a design tool for oligonucleotide; (ii) an analysis tool for performance test; and (iii) a documentation tool for performance test.

The additional components of the tool kit are described below.

Design Tool

The design tool for the oligonucleotide may include software that automatically designs a set of candidate oligonucleotides for detection of the target nucleic acid.

According to an embodiment, the software includes an algorithm for determining a region (e.g., an amplification region) of the target nucleic acid. Most target nucleic acids, particularly from RNA virus, contain high sequence variations. Thus, to detect the target nucleic acids with various sequence variations using the minimum number of oligonucleotides, it is needed to determine a conserved region in multiple target nucleic acids with sequence variations. The software includes an algorithm for determining a conserved region of the target nucleic acids, i.e., the unique region of the target nucleic acids.

According to an embodiment, the software includes the followings:

(i) an algorithm for automatically design candidate sequences for each of oligonucleotides necessary for detecting a target nucleic acid, thereby generating a pool of candidate sequences for each of oligonucleotides; (ii) an algorithm for automatically combining a candidate sequence in a pool with a different candidate sequence in other pool, thereby generating a set of candidate oligonucleotides; and (iii) an algorithm for automatically combining a set of candidate oligonucleotides with a different set of oligonucleotides, thereby generating a combination of sets of candidate oligonucleotides for detection of multiple target nucleic acids.

In the case of designing oligonucleotides (e.g., forward primers, reverse primers, and probes) for detecting the two target nucleic acids, the three algorithms may be performed as follows:

For example, the algorithm for automatically generating a pool of candidate sequences for each oligonucleotide designs a pool of candidate sequences of 1 to 100 forward primers, a pool of candidate sequences of the same or different number of reverse primers, and a pool of candidate sequence pool of the same or different number of probes for detection of a first nucleic acid as well as designs pools of candidate sequences for forward primers, reverse primers, and probes in a similar manner for a second target nucleic acid.

The algorithm for automatically generating a set of candidate oligonucleotides for each target nucleic acid combines 100 candidate sequences in a pool of forward primers, 100 sequences in a pool of reverse primers, and 100 sequences in a pool of probes to generate a set of 10⁶ (100*100*100) candidate oligonucleotides for a first target nucleic acid and repeats the same process to generate a set of 10⁶ candidate oligonucleotides for a first target nucleic acid.

The algorithm for automatically generating a combination of sets of candidate oligonucleotides combines a set of 10⁶ (100*100*100) candidate oligonucleotides for a first target nucleic acid with a set of 10⁶ (100*100*100) candidate oligonucleotides for a second target nucleic acid to generate 10¹² (10⁶*10⁶) combination of sets of candidate oligonucleotides for the first and second target nucleic acids.

According to an embodiment, the software may further include a scoring algorithm to evaluate a pool of candidate sequences for each oligonucleotide, a set of candidate oligonucleotides for each target nucleic acid, and combinations of sets of candidate oligonucleotides for the plurality of target nucleic acids, as described above. The scoring may be performed based on several characteristics of the designed oligonucleotide, e.g., the sensitivity, specificity, coverage, dimer formation, length, Tm value, GC content, and hairpin structure formation, free-energy value. For example, the software scores 100 candidate sequences in a pool of forward primers, 100 candidate sequences in a pool of reverse primers, and 100 candidate sequences in a pool of probes for detection of the first target nucleic acid and performs the same process on the candidate sequences in pools of forward primers, reverse primers, and probes for detection of the second target nucleic acid sequence. The score for each candidate sequence in pools obtained by the process may be used to select proper candidate sequences from each pool to reduce the number of candidate sequences. For example, where 10 sequences are selected from 100 candidate sequences in a pool of the forward primer based on the scores, and 10 sequences are selected in a similar manner on reverse primers and probes to detect the first target nucleic acid, the number of sets of candidate oligonucleotides for the first target nucleic acid is reduced to 10³ (=10*10*10). The scoring-based selection may also be performed on the set of candidate oligonucleotides for each target nucleic acid and on the combination of sets of candidate oligonucleotides for the plurality of target nucleic acids. The scoring-based selection may reduce the number of oligonucleotides necessary for detection of a target nucleic acid, thereby significantly enhancing the driving speed of the software.

Analysis Tool

The analysis tool for the performance test may include software for automatically converting raw performance data into processed performance data, so as to more easily obtain performance information from the raw performance data.

Generally, analysis and interpretation of the raw performance data obtained from a performance test requires considerable skills and may cause significant deviations and errors per person. Thus, the analysis tool according to the disclosure allows the developer and technology provider to obtain oligonucleotide performance information easily, quickly, and precisely by automatically converting the raw performance data into processed performance data by a predetermined algorithm.

As used herein, the term “processed performance data set” may denote a data set obtained by mathematically processing the raw performance data, e.g., a first, second, and third derivative data set of raw performance data.

According to an embodiment, the software included in the analysis tool for the performance test includes an algorithm for automatically subtracting the background of the raw performance data, e.g., the background subtraction algorithm as disclosed in WO 2016/052991, the disclosure of which is incorporated by reference herein in its entirety.

According to an embodiment, the software included in the analysis tool for the performance test includes an algorithm for calibrating the raw performance data, e.g., the algorithm to obtain calibrated data by applying a normalization coefficient to signal values, as disclosed in WO 2017/086762, the disclosure of which is incorporated by reference herein in its entirety.

According to an embodiment, the software included in the analysis tool for the performance test includes an algorithm for automatically fitting the raw performance data into a sigmoid function, e.g., the sigmoid fitting and analysis algorithm disclosed in WO 2019/066572, the disclosure of which is incorporated by reference herein in its entirety.

According to an embodiment, the software included in the analysis tool for the performance test includes an algorithm for automatically extracting the value by which positive/negative may be determined, from the background-subtracted data or fitted function. Examples of the values for determining positive/negative may include, but are not limited to, Ct, delta Ct, melt Tm, and melt peak.

According to an embodiment, the analysis tool for the performance test includes an algorithm for extracting only data for each target nucleic acid from the data obtained at a plurality of different temperatures for the plurality of target nucleic acids, e.g., the algorithms disclosed in WO2015/147370, WO2015/147412, WO2015/147382, and WO2016/093619, the disclosures of which are incorporated by reference herein in their entireties.

According to an embodiment, the analysis tool for the performance test includes software for automatically setting (or optimizing) values for parameters that may cause a false positive or false negative. Various parameters may be used to automatically convert the raw performance data into processed performance data. However, use of fixed parameter values may cause a false positive or false negative in some reaction. Thus, the software finds a parameter that causes a false positive or false negative and automatically converts the parameter value.

According to an embodiment, parameters to be set by the software include at least one of the following: a threshold to determine Ct; a fitting start cycle and a final cycle used in subtracting a background; a reference value and a reference cycle, where the algorithm disclosed in WO 2017/086762 is adopted; R², a maximum slope of the sigmoid fitting curve, and a difference between a maximum and minimum signal values, where the sigmoid fitting algorithm is used; a reference value, where any one of the algorithms disclosed in WO2015-147370, WO 2015/147412, WO2015/147382 and WO2016/093619 is adopted.

Documentation Tool

The documentation tool for the analyzed test data may include software for automatically converting the performance data obtained by the above-described analysis tool into a designated format of document such that the developer or technology provider attending the collaborative development view the performance data in a more intuitive and simplified manner.

Since the performance data obtained by the analysis tool is distributed in various storage spaces in the software, the retrieval/review of the whole data takes considerable time and efforts. As the documentation tool according to the disclosure automatically integrates the distributed performance data into a single document, the performance of oligonucleotides may be easily determined. Such documentation tool facilitates communication between the developer and the technology provider, enhancing collaborative development of the reagent for detection of the target nucleic acid.

According to an embodiment, the documentation tool includes an algorithm that extracts only necessary specific performance information among the processed performance data and copies the extracted performance information to a desired area of the document.

In the disclosure, the development tool kit may further include other various tools, e.g., an encryption supporting tool, than the above-described ones. The tools to be included in the development tool kit may be varied depending on the amount or extent of technology provided by the technology provider. For example, a first technology provider may only provide the performance test tool and the analysis tool, a second technology provider may provide the performance test tool and the design tool, and a third technology provider may provide the performance test tool, design tool, analysis tool, and documentation tool.

In this step, access to the development tool kit by the technology provider for collaborative development of the developer terminal may be performed in various manners.

As used herein, the term “access” used in connection with the tool kit means contacting and using the tool kit in various fashions. The access may include reading, viewing, modifying, copying, or deleting. The access may be controlled. For example, access to the tool kit may be read-only access, meaning that the developer is only allowed to read or view, but not modify, copy, or delete, the tool kit.

In the disclosure, the development tool kit of the reagent for detection of the target nucleic acid is provided from the technology provider terminal at any time.

The development tool kit of the reagent for detection of the target nucleic acid may be received from the technology provider terminal before the request for collaborative development, or the development tool kit may be received from the technology provider terminal in response to the request for collaborative development provided to the technology provider terminal.

According to an embodiment, the development tool kit of the reagent for detection of the target nucleic acid is received by the collaborative development system before the request for collaborative development. For example, before the request for collaborative development, a first development tool kit is received from a first technology provider, and a second development tool kit is received from a second technology provider. Thereafter, when the request for collaborative development is received, the developer terminal is allowed to access a first development tool kit from the first technology provider selected.

According to another embodiment, the development tool kit of the reagent for detection of the target nucleic acid is received in response to the request for collaborative development. For example, if the request for collaborative development is received, the request for collaborative development is transmitted to all technology providers, and the development tool kit is provided from one or more technology providers who are selected from among one or more technology providers responded to the request. As another example, if the request for collaborative development is received, the request for collaborative development is transmitted to the one or more selected technology providers, and the development tool kit is received from the technology provider.

According to an embodiment, access to the development tool kit includes the case where the developer connects to the collaborative development system and directly uses the tool kit served in the form of a cloud or the case where the developer connects to the collaborative development system and downloads, installs, and uses the stored tool kit on the developer terminal.

According to an embodiment, access to the development tool kit includes the case where the developer directly uses the tool kit stored in the technology provider terminal or the case where the developer connects to the technology provider terminal and downloads and uses the stored tool kit on the developer terminal. The direct use or download-and-use of the tool kit stored in the technology provider terminal by the developer may be controlled by the collaborative development system.

The control of the use of the tool kit by the collaborative development system may be done by providing the technology provider terminal with a URL from which the tool kit may be downloaded, providing the technology provider terminal with an ID/PW for accessing the URL, or providing the technology provider terminal with an authority for downloading and then installing the tool kit.

Thus, the technology provider's development tool kit may be stored in the collaborative development system or in the technology provider terminal.

Access to the development tool kit may be controlled. The method according to the disclosure may further include providing the developer terminal with an authority for access to the development tool kit in response to the request for collaborative development.

The development tool kit may be offline accessed. The offline access may be done via various portable storage devices, such as an external hard drive, USB drive, or CD.

Step (c): Receiving of Result from Developer Terminal

Next, the result obtained from using the development tool kit is received from the developer terminal (S400). The result includes performance data for the oligonucleotide.

The developer performs the oligonucleotide development test using the development tool kit accessed in step (b) to generate the result and, in the instant step, the collaborative development system receives the result from the developer terminal.

The range of development test performed by the developer may be varied depending on the range of collaboration between the developer and the technology provider, e.g., an agreement therebetween.

In an example development test, the developer determines the region of the target nucleic acid to be detected. The determination of the target nucleic acid region includes determining a conservative site in the target nucleic acid. The determination of the target nucleic acid region may be performed using, or without using, the design tool provided by the technology provider.

In an example development test, the developer generates a candidate sequence pool for each of a plurality of oligonucleotides for the target nucleic acid sequence. The generation of the candidate sequence pool may be performed using, or without using, the design tool provided by the technology provider.

In an example development test, the developer generates a candidate sequence set for each target nucleic acid by combining a candidate sequence in any pool with a candidate sequence in other pool. The generation of the candidate sequence set may be performed using, or without using, the design tool provided by the technology provider.

In an example development test, the developer generates a candidate sequence set for a plurality of target nucleic acids by combining a candidate sequence set for any target nucleic acid with a candidate sequence set for other target nucleic acid. The generation of the candidate sequence set may be performed using, or without using, the design tool provided by the technology provider.

In an example development test, the developer scores the candidate sequence pool for each oligonucleotide, the candidate sequence set for each target nucleic acid, and the candidate sequence set for the plurality of target nucleic acids and selects some candidate sequence sets based on the scoring. The scoring and selection may be performed using, or without using, the design tool provided by the technology provider.

In an example development test, the developer tests the performance of the oligonucleotide using the performance test tool provided by the technology provider.

In an example development test, the developer obtains processed performance data by analyzing the raw performance data and obtains the performance information therefrom. The analysis of the raw performance data may be performed using, or without using, the analysis tool provided by the technology provider.

In an example development test, the developer documentates the performance information. The documentation may be performed using, or without using, the documentation tool provided by the technology provider.

The developer may optionally use a nucleic acid amplifying device in the development test.

As used herein, the term “nucleic acid amplifying device” is intended to encompass an amplification reaction vessel as well as an amplification reaction device including a thermometer and a detector.

The nucleic acid amplifying device includes various well-known devices capable of temperature adjustment for an amplification reaction. Examples of the device include, but are not limited to, CFX (Bio-Rad), iCycler (Bio-Rad), LightCycler (Roche), StepOne (ABI), 7500 (ABI), ViiA7 (ABI), QuantStudio (ABI), AriaMx (Agilent), and Eco (Illumina).

The amplification reaction vessel includes a tube, a strip, a plate, or other various types of vessels.

The result received from the developer terminal varies depending on the tool included in the development tool kit.

According to the method of the disclosure, when the development tool kit in step (b) includes a performance test tool for the oligonucleotide contained in the reagent for detection of the target nucleic acid and, the result in step (c) includes, at least, the oligonucleotide performance data.

Where the development tool kit in step (b) further includes one or more of the (i) the design tool for oligonucleotide; (ii) the analysis tool for performance test; and (iii) the documentation tool for performance test, the result in step (c) includes data corresponding to the tool.

The result received from the developer terminal is described below in detail.

Design Information about Oligonucleotide

The result received from the developer terminal may include design information about the oligonucleotide.

According to an embodiment, the oligonucleotide design information includes information about the candidate sequence pool of each oligonucleotide, the candidate sequence set of oligonucleotide for each target nucleic acid, or oligonucleotide candidate sequence set for a plurality of target nucleic acids.

Specifically, the oligonucleotide design information includes information about the candidate oligonucleotide used to detect a plurality of target nucleic acids. For example, the oligonucleotide information includes information about a candidate oligonucleotide (e.g., a first forward primer, a first reverse primer, a first probe) for detecting a first target nucleic acid sequence and information about a candidate oligonucleotide (e.g., a second forward primer, a second reverse primer, a second probe) for detecting a second target nucleic acid sequence. Two or more of each of forward primer, reverse primer, and probe may be provided to detect each target nucleic acid sequence.

According to an embodiment, the oligonucleotide design information includes an indication of the sequence or length of the candidate oligonucleotide.

According to an embodiment, the oligonucleotide design information includes an indication, e.g., name or abbreviation, for distinguishing the candidate oligonucleotide.

According to an embodiment, the oligonucleotide design information includes the hybridization position of the candidate oligonucleotide, i.e., the start nucleotide and last nucleotide position of the site where the oligonucleotide is hybridized with the target nucleic acid.

According to an embodiment, the oligonucleotide design information includes the size of an amplicon to be generated by the use of the candidate oligonucleotide.

According to an embodiment, the oligonucleotide design information includes the Tm of the candidate oligonucleotide.

Oligonucleotide Performance Data

The result received from the developer terminal includes oligonucleotide performance data. Performance data means data generated in various tests to examine the performance of oligonucleotide performed by the developer.

The oligonucleotide performance data may include the raw data set, processed data set, or documentated data set.

According to an embodiment, the performance data is raw data that was not subjected to mathematical processing. The raw data indicates the data (output) directly obtained from a device, e.g., a real-time PCR device. The data includes a plurality of data points. As used herein, the term “data point” means a coordinate value including the cycle and signal value. Data points obtained by a signal-generation process, particularly amplification reaction may be displayed with coordinate values that may be shown in the two-dimensional rectangular coordinate system. In the coordinate values, the X axis denotes the number of cycles, and the Y axis denotes the signal value measured or processed in the cycle. The raw data is obtained from using the performance test tool provided by the technology provider.

In another embodiment, the performance data is processed performance data. The processed performance data is baseline subtracted data to remove the background signal value of the raw data. The baseline subtracted data set may be obtained by various methods known in the art (see U.S. Pat. No. 8,560,247). The processed performance data is obtained using, or without using, the analysis tool provided by the technology provider.

In another embodiment, the performance data is documentated performance data.

The documentated performance data is obtained using, or without using, the documentation tool provided from the technology provider.

Information about Selected Oligonucleotide

The result received from the developer terminal includes information about the selected oligonucleotide.

The information about the selected oligonucleotide includes information about an optimal combination of sets of oligonucleotides, which are selected among candidate combinations of sets of oligonucleotides (generated using, or without using, the design tool) for a plurality of target nucleic acids, considering the performance (performance data) of the oligonucleotides.

The selection of a proper oligonucleotide from among candidate oligonucleotides is performed based on the performance, e.g., sensitivity or specificity, of the oligonucleotide.

The result received from the developer terminal is stored in the collaborative development server in the collaborative development system.

The results received from the developer terminal may be identified and classified via user identifiers.

In the disclosure, the developer terminal may include the development device connected with the developer terminal via a network. Thus, the results in this step include those received from the development device.

Step (d): Reviewing of Result

Next, the technology provider terminal and/or the monitoring module of the collaborative development system is allowed to review the received result (S500).

According to an embodiment, the reviewing of the received result is performed by the technology provider terminal. In other words, the received result is provided to the technology provider terminal and reviewed by the technology provider. In relation thereto, the method according to the disclosure may further include providing an authority for access to the received result to the technology provider terminal to allow the technology provider to review the result obtained from using the development tool kit.

The access to the received result by the technology provider terminal may be performed in various manners known in the art. As an example, the access to the received result by the technology provider terminal may be performed by assigning a user identifier to the technology provider terminal and allowing the technology provider terminal to access to the result via authentication of the identifier. As another example, the access to the result by the technology provider terminal may be performed by encrypting the result and allowing the technology provider to decrypt the result.

According to another embodiment, the reviewing of the received result is performed by the monitoring module of the collaborative development system.

In the disclosure, “reviewing” or “monitoring” denotes a process for confirming the received result, e.g., performance data. The confirmation of the performance data includes the confirmation of the accuracy, acceptability, suitability, reliability, robustness, or significance of the performance data.

Specifically, the reviewing by the technology provider includes confirming whether the developer has correctly used the development tool kit provided from the technology provider, i.e., whether he observed instructional information and whether the developer has caused any test error. Further, the reviewing by the technology provider includes the technology provider's feedback to correct an error that arises in the test.

Even when the developer performs the test according to the technology provider's guidelines using software, devices, and instructional information included in the development tool kit, false results may be caused by, e.g., an error in the tool kit provided from the technology provider or a test error caused by the developer. Thus, the technology provider may serve to review the test result by the developer, analyze the cause of an error, and provides a solution to the error.

According to an embodiment, the receiving of the result from the developer terminal and the reviewing of the result by the monitoring module or by the technology provider terminal are repeatedly performed for each test. The oligonucleotide performance test may be performed several times, and the reception and reviewing of the performance test result may be repeated as necessary.

Step (e): Access Review Product

Lastly, the developer terminal is allowed to access the review product (S600).

According to an embodiment, the access to the review product includes allowing the developer to connect to the collaborative development system via the developer terminal and directly view the review product stored in the collaborative development system or allowing the developer to connect to the collaborative development system via the developer terminal, download the review product into the developer terminal, and view the review product stored in the developer terminal.

According to an embodiment, the access to the review product includes allowing the developer to directly view the review product stored in the technology provider terminal or allowing the developer to connect to the technology provider terminal via the developer terminal, download the review product into the developer terminal, and view the review product stored in the developer terminal. In this case, the direct view or download-and-view of the review product stored in the technology provider terminal by the developer may be controlled by the collaborative development system.

Thus, the technology provider's review product may be stored in the collaborative development system or in the technology provider terminal.

The access to the review product may be controlled. The method according to the disclosure may further include providing an authority for the developer terminal to access the review product to the developer terminal.

The review product accessed in the instant step may include an indication of reperforming or approval of the performance test. The reperforming of the performance test may include instructions for (i) redesigning oligonucleotides; (ii) changing enzymes; or (iii) adjusting reaction conditions. Additionally, a modified guideline for reperforming the performance test may be provided.

The collaborative development method according to the disclosure is completed after receiving the result of performance test, reviewing the performance test result by the technology provider, and identifying the review product by the developer.

After the collaborative development is completed, a completion result is generated by the technology provider and/or collaborative development system. The completion result includes information about the feasibility of production of the reagent, such as the performance, marketability, competitivity, cost, and sales profit of the reagent.

The method according to the disclosure may further include providing the completion result to the evaluator terminal to evaluate it and receiving the evaluation result from the evaluator terminal.

Once collaborative development system receives the evaluation result, it registers the collaborative development completion to terminate the collaborative development process.

After registering the collaborative development completion, the collaborative development system may provide a notification for collaborative development completion to the requester terminal, developer terminal, and/or technology provider terminal, as necessary. In this case, the collaborative development completion notification provided to the developer terminal may include the evaluation result.

If the evaluation result of the feasibility for the reagent is good, the method according to the disclosure may further include transmitting a request for producing the reagent for detection of the target nucleic acid to a manufacturer terminal (or the technology provider terminal). The manufacturer may be the technology provider terminal or developer who has attended the collaborative development or may be other third party.

In contrast, if the evaluation result of the feasibility for the reagent is not so good, the method according to the disclosure may abandon the production of the reagent for detection of the target nucleic acid or may request for reevaluation.

According to the disclosure, the collaborative development system may be configured to implement a plurality of collaborative developments. The collaborative development system according to the disclosure includes a subject management module to manage the plurality of collaborative developments, and the subject management module allows each collaborative development to be performed according to steps (a) to (e) described above.

Through the method of the present invention described above, an oligonucleotide to be included in the reagent is selected, and accordingly, a reagent for detecting the target nucleic acid is developed.

III. System for Collaborative Development of Reagent for Detecting Target Nucleic Acid

In another aspect of the disclosure, there is provided a system for collaborative development of a reagent for detection of the target nucleic acid using one or more programs stored in a memory and configured to be executed by a processor.

The one or more programs include instructions executed by the processor to perform:

receiving from a requester terminal a request for collaborative development of a reagent containing an oligonucleotide for detection of a target nucleic acid, wherein the request for collaborative development includes feature information about the target nucleic acid to be detected using the reagent;

allowing a developer terminal to access a technology provider's development tool kit, wherein the development tool kit includes a performance test tool for the oligonucleotide to be included in the reagent, the performance test tool including one or more of software, a device, and instructional information;

receiving from the developer terminal a result obtained from using the development tool kit, wherein the result includes performance data for the oligonucleotide;

allowing the received result to be reviewed by the technology provider terminal or by a monitoring module of a collaborative development system; and

allowing the developer terminal to access the review product, such that an oligonucleotide to be included in the reagent is selected.

Exemplary collaborative development system according to the disclosure is shown in FIG. 2.

The collaborative development system includes a collaborative development server composed of a computer including one or more processors and memories, and instructions stored in a storage medium are configured to operate the collaborative development server.

The collaborative development server includes a controller 110 for controlling each component, a request/evaluation management unit 120 for managing a request for collaborative development and evaluation, a collaborative management unit 130 for substantially managing collaborative development, and a database unit 140 for storing test data and results obtained via collaborative development. Each component is discussed more fully elsewhere in the specification.

IV. Storage Medium for Collaborative Development of Reagent for Detecting Target Nucleic Acid

In still another aspect of the disclosure, there is provided a non-transitory computer-readable storage medium storing instructions executed by one or more processors for collaborative development of a reagent for detection of the target nucleic acid.

When executed by the one or more processors, the instructions enable the computing device to perform:

receiving from a requester terminal a request for collaborative development of a reagent containing an oligonucleotide for detection of a target nucleic acid, wherein the request for collaborative development includes feature information about the target nucleic acid to be detected using the reagent;

allowing a developer terminal to access a technology provider's development tool kit, wherein the development tool kit includes a performance test tool for the oligonucleotide to be included in the reagent, the performance test tool including one or more of software, a device, and instructional information;

receiving from the developer terminal a result obtained from using the development tool kit, wherein the result includes performance data for the oligonucleotide;

allowing the received result to be reviewed by the technology provider terminal or by a monitoring module of a collaborative development system; and

allowing the developer terminal to access the review product, such that an oligonucleotide to be included in the reagent is selected.

V. Computer-Implemented Method for Collaborative Development of Reagent for Detecting Region-Specific Target Nucleic Acid

In still another aspect of the disclosure, there is provided a computer-implemented method for collaborative development of a reagent for detection of a target nucleic acid by a collaborative development system, comprising:

receiving collaborative development application information including feature information about a target nucleic acid;

determining a disease region where a disease associated with the target nucleic acid breaks out using the received feature information about the target nucleic acid;

selecting a developer located in the disease region; and

receiving from the developer terminal a result of performing a test for developing the reagent for detection of the target nucleic acid by the selected developer via a technology provider's support.

For a detailed description of each step, refer to elsewhere in the specification.

According to an embodiment, the requester terminal, the developer terminal, the technology provider terminal, and/or the evaluator terminal denote devices corresponding to desktop computers, cellular devices or smartphones, personal digital assistants (PDAs), laptop computers, or tablet devices, which are capable of providing network connectivity and processing resources to communicate with the collaborative development system over one or more networks. The requester terminal, developer terminal, technology provider terminal, and/or evaluator terminal each may operate a designated service application (e.g., a collaborative development application) configured to communicate with the collaborative development system. The developer terminal may be connected with the development device to share or generate data transmitted/received from the collaborative development system.

One or more examples described herein provide that the methods, techniques, and operations performed by a computing device are performed in a programming scheme or as a computer-implemented method. As used herein, program means use of code or computer-executable instructions. The instructions may be stored in one or more memory resources of a computing device. The steps performed in a programming scheme may, or may not, be automatically performed.

One or more examples described herein may be implemented using a server, a unit, or a module. The server, unit, or module may include a software or hardware component capable of performing part of a program or one or more tasks or functions mentioned herein. As used herein, the unit or module may be present on a hardware component independently from other unit or module. Alternatively, the unit or module may be a shared element or process of other unit, module, program, or device.

One or more examples described herein may be implemented using instructions executable by one or more processors. The instructions may be transferred on a computer-readable storage medium. The collaborative development system shown and described with reference to the drawings provides examples of computer-readable storage media for storing and/or executing instructions for implementing the examples described herein and processing resources. In particular, the collaborative development system shown as an example described herein includes various types of memories for retaining a process(s), data, and instructions. Examples of the computer readable storage media include permanent memory storage devices, such as hard drives in personal computers or servers. Other examples of the computer readable storage media include portable storage units, such as CD or DVD units, flash memories, e.g., those included in smartphones, multi-functional devices, or tablets, and magnetic memories. Computers, terminals, and networking devices (e.g., mobile devices such as cell phones) are examples of severs and devices that utilize the instructions stored on processors, memories, and computer readable storage media. Such examples may also be implemented in the form of computer programs or computer-available transmission media capable of transferring such programs.

The examples described herein are related to use of the collaborative development system for implementing the technology described in the disclosure. According to an embodiment, this technology is performed by the collaborative development system in response to the process executing one or more sequences of one or more instructions included in the memory. The instructions may be read from other machine-readable medium, such as a storage medium, to the memory. The execution of the instruction sequence included in the memory enables the process steps described herein to be carried out. In an alternative implementation, hardwired circuitry along with software instructions may be used instead of the software instructions so as to implement the examples described herein. Thus, the described examples are not limited to any specific combination of the hardware circuitry and software.

The examples described herein may be expanded to individual elements and concepts described herein, independently from other concepts, ideas, or systems and may be combined with elements cited anywhere in the disclosure. Although some examples have been described in detail with reference to the accompanying drawings, the concept is not limited to such examples. Thus, the scope of the concept is intended to be defined by the appended claims and their equivalents. Further, specific features described individually or as some examples may be combined with other features described individually or other examples although not specifically mentioned for the specific features. Thus, the absence of a description of such combination should not be interpreted as excluding such combination from the scope of the disclosure.

1. A computer-implemented method for collaborative development of a reagent for detection of a target nucleic acid by a collaborative development system, comprising:
receiving from a requester terminal a request for collaborative development of a reagent containing an oligonucleotide for detection of a target nucleic acid, wherein the request for collaborative development includes feature information about the target nucleic acid to be detected using the reagent;
allowing a developer terminal to access a technology provider's development tool kit, wherein the development tool kit includes a performance test tool for the oligonucleotide to be included in the reagent, the performance test tool including one or more of software, a device, and instructional information;
receiving from the developer terminal a result obtained from using the development tool kit, wherein the result includes performance data for the oligonucleotide;
allowing the received result to be reviewed by the technology provider terminal or by a monitoring module of a collaborative development system; and
allowing the developer terminal to access the review product, such that an oligonucleotide to be included in the reagent is selected.

2. The computer-implemented method of claim 1, wherein the developer terminal is a terminal of a developer who has already obtained or is able to obtain a clinical sample containing, or suspected of containing the target nucleic acid.

3. The computer-implemented method of claim 2, wherein the result obtained from using the development tool kit includes a result of test for the obtained clinical sample.

4. The computer-implemented method of claim 1, wherein the developer terminal is a terminal of a developer who is located in a region where a disease associated with the target nucleic acid is breaking out or has broken out.

5. The computer-implemented method of claim 1, wherein the requester terminal is identical to the developer terminal.

6. The computer-implemented method of claim 1, wherein the reagent is for detecting a plurality of target nucleic acids.

7. The computer-implemented method of claim 1, wherein the feature information about the target nucleic acid includes: (i) a name of an organism containing the target nucleic acid; (ii) a name of a gene containing the target nucleic acid; (iii) a sequence of the target nucleic acid; (iv) an intended use of the reagent; (v) a name of a disease to be detected with the reagent; and/or (vi) a classification symbol of the disease to be detected with the reagent.

8. The computer-implemented method of claim 1, further comprising transmitting a request for evaluation to an evaluator terminal in response to the request for collaborative development and receiving an evaluation result of the feasibility of collaborative development from the evaluator terminal.

9. The computer-implemented method of claim 1, further comprising evaluating the feasibility of collaborative development by an evaluation management module of the collaborative development system in response to the request for collaborative development.

10. The computer-implemented method of claim 1, wherein the evaluator terminal includes a technology provider terminal.

11. The computer-implemented method of claim 1, wherein the developer terminal is a terminal of a developer who is selected from a plurality of candidate developers.

12. The computer-implemented method of claim 1, wherein the development tool kit is provided from a technology provider selected from a plurality of candidate technology providers.

13. The computer-implemented method of claim 1, wherein any one or more of the device, the software, or the instructional information included in the performance test tool are used for determination of a sensitivity or specificity of the oligonucleotide.

14. The computer-implemented method of claim 13, wherein the device for determining the sensitivity or specificity of the oligonucleotide includes one or more devices selected from the group consisting of a nucleic acid extracting device, a nucleic acid dispensing device, and a nucleic acid detecting device.

15. The computer-implemented method of claim 13, wherein the software for determining the sensitivity or specificity of the oligonucleotide includes an instruction for dispensing (localizing) the oligonucleotide in a designated position of a reaction vessel.

16. The computer-implemented method of claim 13, where the instructional information for determining the sensitivity or specificity of the oligonucleotide includes processes and conditions for the performance test of the oligonucleotide.

17. The computer-implemented method of claim 1, wherein the development tool kit further includes one or more of:
(i) a design tool for the oligonucleotide;
(ii) an analysis tool for a performance test; and
(iii) a documentation tool for the performance test.

18. The computer-implemented method of claim 17, wherein the design tool for the oligonucleotide includes software that automatically designs a set of candidate oligonucleotides for detection of the target nucleic acid.

19. The computer-implemented method of claim 17, wherein the analysis tool for the performance test includes software that automatically converts raw performance data into processed performance data by a predetermined parameter.

20. The computer-implemented method of claim 17, wherein the analysis tool for the performance test includes software that automatically sets a parameter value to cause a false positive or a false negative.

21. The computer-implemented method of claim 17, wherein the documentation tool for the performance test includes software that automatically converts processed performance data into a designated format of document.

22. The computer-implemented method of claim 1, wherein the receiving of the result from the developer terminal and the reviewing of the result by the monitoring module or by the technology provider terminal are repeatedly performed for each test.

23. The computer-implemented method of claim 1, wherein the review product includes an indication of reperforming or approval of the performance test.

24. The computer-implemented method of claim 1, wherein the collaborative development system is configured to implement a plurality of collaborative developments.

25. A system for collaborative development of a reagent for detection of the target nucleic acid using one or more programs stored in a memory and configured to be executed by a processor, wherein
the one or more programs include instructions executed by the processor to perform:
receiving from a requester terminal a request for collaborative development of a reagent containing an oligonucleotide for detection of a target nucleic acid, wherein the request for collaborative development includes feature information about the target nucleic acid to be detected using the reagent;
allowing a developer terminal to access a technology provider's development tool kit, wherein the development tool kit includes a performance test tool for the oligonucleotide to be included in the reagent, the performance test tool including one or more of software, a device, and instructional information;
receiving from the developer terminal a result obtained from using the development tool kit, wherein the result includes performance data for the oligonucleotide;
allowing the received result to be reviewed by the technology provider terminal or by a monitoring module of a collaborative development system; and
allowing the developer terminal to access the review product, such that an oligonucleotide to be included in the reagent is selected.

26. A non-transitory computer-readable storage medium storing instructions executed by one or more processors for collaborative development of a reagent for detection of the target nucleic acid in a computing device, wherein
when executed by the one or more processors, the instructions enable the computing device to perform:
receiving from a requester terminal a request for collaborative development of a reagent containing an oligonucleotide for detection of a target nucleic acid, wherein the request for collaborative development includes feature information about the target nucleic acid to be detected using the reagent;
allowing a developer terminal to access a technology provider's development tool kit, wherein the development tool kit includes a performance test tool for the oligonucleotide to be included in the reagent, the performance test tool including one or more of software, a device, and instructional information;
receiving from the developer terminal a result obtained from using the development tool kit, wherein the result includes performance data for the oligonucleotide;
allowing the received result to be reviewed by the technology provider terminal or by a monitoring module of a collaborative development system; and
allowing the developer terminal to access the review product, such that an oligonucleotide to be included in the reagent is selected.

27. A computer-implemented method for collaborative development of a reagent for detection of a target nucleic acid by a collaborative development system, comprising:
receiving collaborative development application information including feature information about a target nucleic acid;
determining a disease region where a disease associated with the target nucleic acid breaks out using the received feature information about the target nucleic acid;
selecting a developer located in the disease region; and
receiving from the developer terminal a result of performing a test for developing the reagent for detection of the target nucleic acid by the selected developer via a technology provider's support.
